(19) United States
(12) Patent Application Publication
(10) Pub. No.: US 2025/0182902 A1
(43) Pub. Date: Jun. 5, 2025

(54) SYSTEMS AND METHODS FOR PREDICTING OUTCOMES FOR A LUNG UNDERGOING AN EX VIVO LUNG PERFUSION

(71) Applicant: University Health Network, Toronto (CA)

(72) Inventors: Shafique Keshavjee, Toronto (CA); Bo Wang, Toronto (CA); Marcelo Cypel, Toronto (CA); Andrew Sage, Toronto (CA)

(21) Appl. No.: 18/842,351

(22) PCT Filed: Feb. 28, 2023
(86) PCT No.: PCT/CA2023/050251

§ 371 Date: Aug. 28, 2024

Related U.S. Application Data
(60) Provisional application No. 63/315,042, filed on Feb. 28, 2022.

(51) Int. Cl. G16H 50/30; G06N 20/00; G16H 20/40; G16H 30/40; G16H 50/20

(52) U.S. Cl. CPC G16H 50/30; G06N 20/00; G16H 20/40; G16H 30/40; G16H 50/20

(57) ABSTRACT

Devices, systems and methods for predicting an outcome for a lung undergoing an ex vivo lung perfusion are provided. The device includes a processor configured to: obtain values for a first set of features from data obtained for lung features including donor parameters, physiological parameters, biochemical parameters, and/or biomarkers collected during EVLP; process the data for a subset of the lung features to determine values for a second set of features based on temporal characteristics of the data for the subset of the lung features; and determine predicted probabilities for several outcome classifications by providing the values for the first and second sets of lung features as inputs to a machine learning model.

### CROSS-REFERENCE TO RELATED PATENT APPLICATION

This application claims the benefit of U.S. Provisional Patent Application No. 63/315,042 filed Feb. 28, 2022; the entire contents of Patent Application 63/315,042 is hereby incorporated by reference.

### FIELD

The disclosure pertains to methods, devices and/or systems for assessing and predicting outcomes for post-transplant outcomes of donor lung grafts undergoing an ex vivo lung perfusion (EVLP).

### BACKGROUND

Ex vivo lung perfusion (EVLP) is a novel technique that was developed to prolong the normothermic assessment period of donor organs during lung transplantation. EVLP has been clinically validated and the technique is gaining widespread adoption worldwide. Currently, EVLP is hampered by a lack of making a prediction using biomarkers that serve as reliable markers as to the process of EVLP, or the outcome of the organs that have been subject to EVLP during organ transplantation. Specifically, it is difficult to predict “patient outcome(s)” (PO) after transplant with a lung or lungs having been subject to EVLP. Furthermore, many potential donor organs are placed on EVLP with the hope that they will improve and become suitable for transplant. However, in some cases the status of these lungs may not change and they will ultimately be discarded following EVLP.

Accordingly, there is a need for the development of predictive models that use input features based on suitable biomarkers, and other data for predicting (i) EVLP outcomes and/or (ii) predicting patient outcomes after transplantation of EVLP treated lungs.

### SUMMARY

According to a broad aspect, there is disclosed a method for predicting an outcome for a lung undergoing an ex vivo lung perfusion (EVLP), the method including: obtaining a plurality of perfusate samples taken over a time period during the EVLP; determining levels, optionally concentrations, of biomarkers from the perfusate samples taken over the time period; for one or more of the biomarkers: fitting a time series of the levels of the biomarker with a corresponding biomarker model and determining values for biomarker model parameters that define the corresponding biomarker model based on said fitting; and calculating a prediction of an outcome for an individual who receives the lung with a machine learning model, wherein the values for the biomarker model parameters are used as inputs to the machine learning model and the machine learning model outputs the prediction of the outcome.

The outcome can be suitability for transplant or patient outcome after transplant.

In another aspect, in accordance with the teachings herein is a method for determining if a lung undergoing an EVLP is suitable for transplant, the method including: obtaining a plurality of lung feature measurements (e.g. EVLP data) taken over a time period during the EVLP; determining levels of the lung features taken over the time period; for one or more of the lung features: fitting a time series of the levels of the lung feature with a corresponding lung feature model and determining values for lung feature model parameters that define the corresponding lung feature model based on said fitting; and calculating a prediction of an outcome for an individual who receives the lung with a machine learning model, wherein the values of the lung feature model parameters are used as inputs to the machine learning model and the machine learning model outputs the prediction of whether the donor lung is suitable for transplant.

In another aspect, in accordance with the teachings herein, is a method for determining if a lung undergoing an EVLP is suitable for transplant, the method including: obtaining a plurality of perfusate samples taken over a time period during the EVLP; determining levels, optionally concentrations, of biomarkers from the perfusate samples taken over the time period; for one or more of the biomarkers: fitting a time series of the levels, optionally concentrations, of the biomarker with a corresponding biomarker model and determining values for biomarker model parameters that define the corresponding biomarker model based on said fitting; and calculating a prediction of an outcome for an individual who receives the lung with a machine learning model, wherein the values of the biomarker model parameters are used as inputs to the machine learning model and the machine learning model outputs the prediction of whether the donor lung is suitable for transplant.

In another aspect, in accordance with the teachings herein, is a method for determining if a lung undergoing an EVLP is suitable for transplant, the method including: obtaining a plurality of biomarker parameter measurements taken over a time period during the EVLP; determining levels of biomarkers taken over the time period; for one or more of the biomarkers: fitting a time series of the levels, optionally concentrations, of the biomarker with a corresponding biomarker model and determining values for biomarker model parameters that define the corresponding biomarker model based on said fitting; and calculating a prediction of an outcome for an individual who receives the lung with a machine learning model, wherein the values of the biomarker model parameters are used as inputs to the machine learning model and the machine learning model outputs the prediction of whether the donor lung is suitable for transplant.

In another aspect, in accordance with the teachings herein there is provided at least one embodiment of a method for predicting an outcome for a lung undergoing an ex vivo lung perfusion (EVLP), the method comprising: obtaining EVLP data for measuring at least one lung feature of the lung taken over a time period; measuring values from the EVLP data to obtain at least one time series for the at least one lung feature over the time period; fitting the at least one time series of the at least one lung feature with a corresponding lung feature model, and determining values for lung feature model parameters that define the at least one corresponding lung feature model based on said fitting; and calculating a prediction of an outcome for an individual who receives the lung with a machine learning model, wherein the values for the lung feature model parameters are used as inputs to the machine learning model and the machine learning model outputs the prediction of the outcome.

In at least one embodiment, the at least one lung feature comprises at least one biomarker and the corresponding lung feature model is a corresponding biomarker model.

In such cases where the at least one lung feature comprises at least one biomarker, the method further includes filtering the measured values, optionally concentrations, of the at least one biomarker to account for circuit dilution prior to the step of fitting the time series of the measured values, e.g., levels, optionally concentrations, of the lung features, optionally biomarkers with the lung feature models, optionally biomarker, models.

In at least one embodiment, the method further includes obtaining EVLP data every 1 millisecond, several milliseconds, tens of milliseconds, hundreds of milliseconds, 1 second, 5 seconds, 30 seconds, 1 min, 5 min, 10 min or 15 min.

In at least one embodiment, the method further includes obtaining a perfusate sample every 15 minutes from 0-180 minutes of perfusion.

In at least one embodiment, the biomarkers include GM-CSF, IL-10, IL-1β, IL-6, IL-8, sTNFR1, and/or sTREM1.

In at least one embodiment, the method further includes using standardized EVLP data (e.g., lung feature measurements) optionally perfusate data to correct the measured values, e.g., levels, optionally concentrations of the biomarkers.

In at least one embodiment, the corresponding biomarker model includes a linear model, a quadratic model, an exponential model, a 4PL model, or a 5PL model.

In at least one embodiment, the machine learning model includes a multivariate logistic regression model.

In at least one embodiment, the outcome comprises mechanical ventilation length of time or time to extubation.

In at least one embodiment, the outcome comprises: (a) an EVLP outcome including suitable or unsuitable, (b) a transplant outcome including good patient outcome or bad patient outcome and/or (c) ICU length of stay.

In at least one embodiment, when the donor lung predicated as being likely suitable for transplant the method includes subsequently transplanting the donor lung into the patient.

According to a broad aspect, there is disclosed an electronic device for predicting an outcome, such as suitability for transplant, or a lung transplant patient outcome, for a lung undergoing an ex vivo lung perfusion (EVLP), the system including: one or more user interfaces for receiving user input and providing indication to the user; a memory; and a processor operatively coupled to the memory and the one or more user interfaces. The processor is configured to: determine levels of lung features, optionally biomarkers, over time; for one or more of the biomarkers: fit a time series of the levels of the lung features, optionally biomarkers, with lung feature models, optionally biomarker models, determine a best fit based on said fitting, and determine parameters of the lung feature model, optionally biomarker model corresponding to said best fit; and calculating a prediction of an outcome for an individual who receives the lung with a machine learning model, wherein the lung feature model, optionally the biomarker model, parameters are used as inputs to the machine learning model and the machine learning model outputs the prediction of the outcome.

According to a broad aspect, there is disclosed a method for predicting an outcome for a lung undergoing an ex vivo lung perfusion (EVLP), the method including: obtaining values for a first set of features from data obtained for lung features including one or more donor parameters, one or more physiological parameters, one or more biochemical parameters, and/or one or more biomarker parameters collected during EVLP; processing the data for a subset of the parameters to determine values for a second set of features based on temporal characteristics of the data for the subset of the parameters; and determining predicted probabilities for at least one outcome classification by providing the values for the first and second sets of features as inputs to a machine learning model.

In at least one embodiment, the one or more donor parameters include: age; sex; body mass index (BMI); donor type donation-after-brain-death (DBD); donor total lung capacity (TLC) and/or donation-after-cardiac-death (DCD).

In at least one embodiment, the first set of features also include one or more recipient parameters.

In at least one embodiment, the one or more recipient parameters comprise: recipient age, recipient sex, recipient BMI, recipient status, and/or indication for transplant.

In at least one embodiment, the one or more physiological parameters include: change in oxygen partial pressure (ΔPO2); change in carbon dioxide partial pressure (ΔPCO2); pH; ventilator air flow; dynamic compliance; static compliance; pulmonary artery (PA) & left atrial (LA) pressure; vascular resistance; airway pressure including peak, mean and plateau; positive end-expiratory pressure (PEEP); edema; perfusate loss; and/or +/−perfusate exchange.

In at least one embodiment, the one or more biochemical parameters include: Ca²⁺; Cl⁻; K⁺; Na⁺; base excess; HCO₃⁻; pH; glucose; and/or lactate.

In at least one embodiment, the one or more biomarker parameters include: GM-CSF; IL-10; IL-1β; IL-6; IL-8, sTNFR1, and/or sTREM1.

In at least one embodiment, the first set of features also include one or more recipient parameters.

In at least one embodiment, the one or more recipient parameters comprise: recipient age, recipient sex, recipient BMI, recipient status, and/or indication for transplant.

In at least one embodiment, the one or more temporal characteristics comprise one or more statistical measurements including a minimum value, a maximum value, a last recorded value and/or a trend for the data collected for the subset of the parameters.

In at least one embodiment, values for at least one of the features from the first set of features are determined by; measuring values from the data to obtain at least one time series for the at least one lung feature over a time period; fitting the at least one time series for the at least one lung feature with a corresponding lung feature model, determining values for lung feature model parameters that define the at least one corresponding lung feature model based on said fitting, and providing the values for the lung feature model parameters as input to the machine learning prediction model.

In at least one embodiment, values for at least one of the features from the first set of features are determined by obtaining an x-ray image of the lung, performing image processing on the x-ray image and determining the values from the processed x-ray image.

In at least one embodiment, the machine learning model outputs at least one of one of three-outcome classifications including: (i) lung unsuitable for transplantation; (ii) EVLP transplant resulting in a time to extubation of >72 hours; and (iii) EVLP transplant resulting in a time to extubation of <72 hours.

In at least one embodiment, the machine learning model includes a decision tree algorithm.

In at least one embodiment, the machine learning model includes an extreme gradient boosting (XGBoost) machine learning algorithm.

In at least one embodiment, the machine learning model may determine a relative weighting of the values for the first and second sets of features.

In at least one embodiment, static compliance is the top three weighted features.

In at least one embodiment, the top five weighted features do not include any protein biomarkers.

In at least one embodiment, the machine learning model was trained using k-fold cross-validation.

In at least one embodiment, k may be at least 3.

In at least one embodiment, when the donor lung predicated as being likely suitable for transplant the method includes subsequently transplanting the donor lung into the patient.

According to a broad aspect, there is disclosed an electronic device for predicting an outcome for a lung undergoing an ex vivo lung perfusion (EVLP), the device including: one or more user interfaces for receiving user input and providing indication to the user; a memory; and a processor operatively coupled to the memory and the one or more user interfaces. The processor is configured to: obtain values for a first set of features from data obtained for the lung features including one or more donor parameters, one or more physiological parameters, one or more biochemical parameters, and/or one or more biological parameters collected during EVLP; process the data for a subset of the parameters to determine values for a second set of features based on temporal characteristics of the data for the subset of the parameters; and determine predicted probabilities for several outcome classifications by providing the values for the first and second sets of parameters as inputs to a machine learning model.

In at least one embodiment, the device is configured such that the first set of features also include one or more recipient parameters. Preferably, the one or more recipient parameters comprise: recipient age, recipient sex, recipient BMI, recipient status, and/or indication for transplant.

According to a broad aspect, there is disclosed a computer program product including a computer readable memory storing computer executable instructions thereon that when executed by a computer perform the method steps described in the present subject matter.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a block diagram of an example embodiment of an electronic device for predicting outcomes for a lung undergoing EVLP in accordance with the teachings herein.

FIG. 2 is a block diagram of an example embodiment of a method for predicting outcomes for a lung undergoing EVLP in accordance with the teachings herein.

FIG. 3 is a block diagram of another example embodiment of a method for predicting outcomes for a lung undergoing EVLP in accordance with the teachings herein.

FIG. 4 shows a schematic representation of an example embodiment of a prediction model in accordance with the teachings herein where the prediction model uses features derived from an EVLP circuit (top left) and as well as features derived from biological, physiological, and biochemical assessments (bottom left) as inputs into an XGBoost machine learning algorithm to predict organ suitability for transplant (bottom right).

FIG. 5 is a schematic for retrospective EVLP case review in accordance with at least one of example embodiment of a prediction model described herein.

FIG. 6 shows an example of predictive model results in accordance with at least one embodiment in accordance with the teachings herein where the output from the predictive model shows the likelihood that a donor lung undergoing EVLP is suitable for transplant (top panel) and/or the probability that, if transplanted, a recipient would be extubated in less than 72 hours post-transplant (bottom panel).

FIG. 7 is a schematic of a study overview for investigating fitting different models for different biomarkers.

FIGS. 8A-8G shows time series of various biomarker concentrations (median±95% CI) where the y-axes represent biomarker concentrations and the x-axes represent EVLP duration. Each panel represent different biomarkers as follows: (A) GM-CSF; (B) sTNFR1; (C) sTREM1; (D) IL-10; (E) IL-1β; (F) IL-6; (G) IL-8.

FIGS. 9A and 9B show ROC curves for assessing InsighTx model performance in Study #3 for Test Dataset 1 (FIG. 9A) and Test Dataset 2 (FIG. 9B).

FIG. 10 shows an example of real-time ventilator data obtained during EVLP.

FIG. 11A shows an example of ventilator flow versus time with annotations for three lung assessments performed during EVLP.

FIG. 11B shows an example of dynamic compliance (blue) versus time with annotations for individual breath segments recorded before (“b”), during (“d”), and after (“a”) lung assessments performed during EVLP.

FIGS. 12A-12E show an example breath-by-breath ventilator analysis results.

FIGS. 13A and 13B show an example of mean peak pressure from donor lung breaths during EVLP (FIG. 13A) and mean static compliance (FIG. 13B) in good (TTE<72 hrs) and poor (TTE>72 hrs+declined) outcome groups.

FIG. 13C shows breath-by-breath mean pressure and peak pressure versus time where dots show the plateau pressure from every inspiratory pause performed during EVLP.

FIG. 14 shows an example of pilot real-time data recording in lung perfusate using a porcine model of EVLP.

Further aspects and features of the example embodiments described herein will appear from the following description taken together with the accompanying drawings.

### DETAILED DESCRIPTION OF THE DISCLOSURE

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art.

The term “outcome” as used herein can refer to patient outcome as further defined below, or suitability for transplant.

The term “patient outcome” as used herein means one or more of primary graft dysfunction (PGD) grade, graft-related patient death, total hospital length of stay, transplant-related hospital length of stay, total intensive care unit (ICU) length of stay, transplant-related ICU length of stay, post-transplant ICU length of stay, APACHE score, time to extubation (or days on mechanical ventilation), patient-related use of extracorporeal membrane oxygenation (ECMO).

The term “biomarker” or “biomarker parameters” as used herein means two, three or more of GM-CSF, IL-6 (also referred to as IL6), IL-8 (CXCL8), IL-10 (also referred to as IL10) and IL-1β (also referred to as IL1β or IL1beta) measured in EVLP perfusate (e.g., a perfusate sample), optionally the same perfusate sample or perfusate samples obtained at different times. For example, the biomarkers may comprise 3, 4, 5, 6 or all 7 biomarkers selected from GM-CSF, IL-6, IL-8, IL-10, IL-1β, sTNFR1 and sTREM1.

The term “biochemical parameters” as used herein refers biochemical parameters measured in the EVLP, optionally the same perfusate sample, where said biochemical parameters can include, but are not limited to: base excess, bicarbonate, potassium, sodium, calcium, chloride, glucose, lactate, pH, etc. Base excess for example, is a number derived from the acid-base chemistry of the EVLP perfusate.

The term “physiological parameters” as used herein refers to physiological parameters of the lung (i.e., donor lung), where the physiological parameters can include, but are not limited to: driving pressure, PCO₂ (measured and differential), PO2 (also referred to as gas exchange and including measured and differential), airway pressure, static and dynamic compliance, PA and/or LA pressure, and/or pulmonary vascular resistance, etc. of the donor lung. These parameters can be measured using a ventilator, patient monitor (e.g., GE Dash 3000s connected to the lung/EVLP system and used to monitor pressures during EVLP) or calculated from the outputs of these machines (i.e., subtract two values).

The term lung feature as used herein refers to biomarker parameters, physiological parameters, and biochemical parameters that may be used as part of the inputs that are provided to a machine learning model for predicting an outcome for a lung undergoing an ex vivo lung perfusion (EVLP) and/or predicting if a lung undergoing EVLP is suitable for transplant.

The term “GM-CSF” as used herein means granulocyte-macrophage colony-stimulating factor which is a secreted monomeric glycoprotein, and includes all naturally occurring forms, for example from all species and particularly human including for example human GM-CSF which as amino acid sequence accession P04141, herein incorporated by reference.

The term “IL-6” or “IL6” as used herein means interleukin-6 which is a secreted cytokine, and includes all naturally occurring forms, for example from all species and particularly human including for example human IL-6 which has amino acid sequence accession P05231, which is herein incorporated by reference.

The term “IL-8” also referred to as CXCL8, as used herein means interleukin-8 which is a secreted cytokine, and includes all naturally occurring forms, for example from all species and particularly human including for example human IL-8 which has amino acid sequence accession P10145, which is herein incorporated by reference.

The term “IL-10” or “IL10” as used herein means interleukin-10, which is a secreted cytokine, and includes all naturally occurring forms, for example from all species and particularly human including for example human IL-10 which has amino acid sequence accession P22301, which is herein incorporated by reference.

The term “IL1β”, “IL-1β” or “IL1beta as used herein means interleukin-1β, which is a secreted cytokine, and includes all naturally occurring forms, for example from all species and particularly human including for example human IL-10 which has amino acid sequence accession P01584, which is herein incorporated by reference.

The term “sTNFR1” or “soluble (TNFRSF1A)” used herein means non-cell bound forms of tumor necrosis factor (TNF) receptor superfamily member 1A, and includes all naturally occurring cleaved or released forms, for example from all species and particularly human including for example human sTNFR1 which has at least the extracellular portion of TNFR1, for example amino acid 22 to 211 of accession number P19438, which is herein incorporated by reference.

The term “soluble TREM1” or sTREM-1 as used herein means non-cell bound forms of Triggering receptor expressed on myeloid cells and includes all naturally occurring cleaved or released forms, for example from all species and particularly human including for example human sTREM-1 which has at least the extracellular portion of sTREM-1, for example amino acid 21 to 205 of accession number Q9NP99, which is herein incorporated by reference.

The term “outcome classification” as used herein means a predicted outcome based on the methods and systems herein disclosed. For example, the outcome classifications include (i) unsuitable for transplantation; (ii) EVLP transplant resulting in a time to extubation of >72 hours; and (iii) EVLP transplant resulting in a time to extubation of <72 hours. These outcomes can be used to determine if a lung is suitable for transplant. For example, as indicated in (i), such a lung is unsuitable. A lung classified as (ii) may be suitable but patient outcome is predicted to be different than (iii), whereas a lung classified as (iii) is considered suitable for transplant with for example good patient outcome.

The term “EVLP transplant resulting in a time to extubation of >72 hours” as used herein means lung grafts that are predicted to be and/or which are characterized as being suitable for clinical transplantation after EVLP; and if transplanted in a recipient, are predicted to result in patient extubation more than 72 hours post-transplant.

The term “EVLP transplant resulting in a time to extubation of <72 h hours” as used herein means lung grafts that are predicted to be and/or which are characterized as being suitable for clinical transplantation after EVLP; and if transplanted in a recipient, are predicted to result in patient extubation in less than 72 hours post-transplant.

The term “unsuitable for transplantation” as used herein means lung grafts that are predicted to be and/or which are characterized as being less or unsuitable for clinical transplantation after EVLP or, in the recipient after transplantation, inducing poor outcome such as death from graft-related causes within 30 days, PGD3, requiring extracorporeal life support/ECMO, prolonged hospital/ICU stays, or time on mechanical ventilation. Examples of a poor-PO graft include a graft that after transplanting would result in a patient requiring an extended ICU stay (for example greater than 3 days or greater than two-weeks (14 days)), as well as a graft that has an increased risk of having a PGD3 lung transplant outcome. A lung graft can be characterized as being unsuitable for clinical transplant after EVLP for example after visual and physiological examination such as when gas exchange function is not acceptable represented by a partial pressure of oxygen less than 350 mmHg with a fraction of inspired oxygen of 100%; or 15% worsening of lung compliance compared to 1 h EVLP; or 15% worsening of pulmonary vascular resistance compared to 1 h EVLP; or worsening of ex vivo x-ray. Biomarkers and other lung features and/or EVLP data that are able to predict suitability can provide a more accessible quantitative benchmark for use in assessing transplant suitability.

The term “Acute Physiology And Chronic Health Evaluation Score” or “APACHE score” as used herein refers to an initial risk classification system for severely ill hospitalized patients. For example, it is applied within 24 hours of admission of a patient to an ICU. An integer score is computed based on several measurements, and higher scores correspond to more severe disease and a higher risk of death. For example, the point score is calculated from a patient's age and 12 routine physiological measurements: AaDO2 or PaO2 (depending on FiO2); temperature (rectal); mean arterial pressure; pH arterial; heart rate; respiratory rate; sodium (serum); potassium (serum); creatinine hematocrit; white blood cell count; and Glasgow Coma Scale. The score can also take into account of whether the patient has acute renal failure, and whether prior to hospital admission the patient has severe organ system insufficiency or is immunocompromised.

The term “perfusate sample” as used herein means an aliquot of a perfusion solution such as STEEN Solution™ that is used for EVLP and which is taken subsequent to starting EVLP, for example after at least or at about several seconds, 1, 2, 3, 4, 5, 10, 15, 30, 45, 60, 75, 90 and/or 105 min, and/or after at least or at about 2, 2.5, 3, 3.5, 4, 4.5, 5, 5.5 and/or 6 hours subsequent to starting EVLP, or optionally at time of fluid replenishment or any time between 15 min and 6 hours, optionally between 1 hour and 4 hours, or any increment of 1 minute, 5 minutes or 15 minutes between 0 and 6 hours or any time therebetween. The term “perfusate sample” and “EVLP perfusate sample” are used interchangeably in the present disclosure. Perfusate samples can be used directly or snap frozen for later testing. The perfusate sample can, for example, be purified and/or treated prior to assessment.

The term “lung feature measurement” as used herein means a measurement that is performed on a lung feature. For example, some measurements for lung features may be obtained from perfusate samples. Lung feature measurements can be determined over different time intervals depending on the lung feature being measured. For example, some lung features that are biochemical/physiological parameters can be measured using EVLP data obtained on the order of seconds such as pH, PCO2, and PO2 which may be measured every second, while K+ can be measured every 6 seconds. Other lung features may be measured) on the order of milliseconds and therefore in real-time. Biochemical and physiological parameters can be measured at various time intervals, including at 1 second, 5 seconds, 30 seconds, 1 min, 5 min, 10 min or 15 min, for example after starting EVLP. Measurements for some parameters can be made continuously at a time interval and measured in real time. For example, oxygenation can be measured continuously, for example in second time intervals.

The term “perfusion solution” as used herein means a buffered nutrient solution that can be used for EVLP, including for example STEEN Solution™. STEEN Solution™ is a buffered extracellular solution developed specially for EVLP that contains Dextran 40, human serum albumin and extracellular electrolyte composition (low K+) that provides cellular/organ protection and optimized colloid osmotic pressure. The skilled person can readily recognize that the perfusion solution can be any buffered nutrient solution that is suitable for and/or supports ex vivo lung perfusion for lungs that may be used for transplantation.

The term “declined lungs” as used herein means lungs that after EVLP are determined to be unsuitable for transplant.

The term “suitability for transplant” as used herein means an organ that is predicted to be a good outcome lung graft, for example to have a decreased risk of a prolonged ICU (e.g., greater than 3 days, greater than 14 days) stay post-transplant. For example, a lung that would be predicted to involve 3 days or less of ICU stay for the recipient, would be considered a particularly suitable lung for transplant. A lung that would be predicted to involve 14 days or less of ICU stay for the recipient, may be considered a suitable lung for transplant.

The term “PGD3” as used herein means Primary Graft Dysfunction Grade 3 as defined by the standardized consensus criteria of International Society for Heart and Lung Transplantation (ISHLT) or similar

The term “EVLP data” as used herein may refer to data related to an EVLP procedure, including, for example: data related to biomarker parameters (e.g. levels such as concentrations, trends, rates, rate of change, time dependent change etc.); biochemical parameter values (e.g. levels such as concentrations, trends, rates, rate of change, time dependent change, etc.) including for example base excess, bicarbonate, potassium, sodium, calcium, chloride, glucose, lactate and pH and; physiological parameter values including driving pressure, PCO2 (measured and differential), PO2 (measured and differential), airway pressure, static and dynamic compliance, PA and/or LA pressure, and pulmonary vascular resistance (e.g. levels such as concentrations, trends, rates, rate of change, time dependent change, etc.); data related to features and timing of the EVLP procedure, including time after start of EVLP perfusate sample obtained; and donor data.

The term “donor data” can refer to for example but not limited to donor characteristics, for example gender, type (DBD or DCD), age, body mass index (BMI), and/or smoking history.

The term “subject” as used herein includes all members of the animal kingdom including mammals, and suitably refers to humans.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. More specifically, the terms “substantially”, “about” and “approximately” may mean plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”, if this does not result in a substantial change in outcome associated with the numerical range. For example, the term “about” may mean a deviation of about +/−0.5%, +/−1%, +/−2%, +/−5%, +/−10% or even +/−15% to the number to which the word “about” applies.

Further, the definitions and embodiments described in particular sections are intended to be applicable to other embodiments herein described for which they are suitable as would be understood by a person skilled in the art. For example, in the following passages, different aspects of the disclosure, are defined in more detail. Each aspect so defined may be combined with any other aspect or aspects unless clearly indicated to the contrary. In particular, any feature indicated as being preferred or advantageous may be combined with any other feature or features indicated as being preferred or advantageous as long as it results in an operable combination (e.g., works properly and provided utility).

Various embodiments in accordance with the teachings herein will be described below to provide examples of at least one embodiment of the claimed subject matter. No embodiment described herein limits any claimed subject matter. The claimed subject matter is not limited to methods, devices, or systems having all of the features of any one of the methods, devices, or systems described below or to features common to multiple or all of the methods, devices, or systems described herein. It is possible that there may be a method, device, or system described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

A portion of the example embodiments of the methods, systems, or devices described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including volatile memory, non-volatile memory and/or at least one storage device). These devices may also have at least one input element (e.g., a keyboard, a mouse, a touchscreen, and the like) and at least one output element (e.g., a display screen, a printer, a wireless radio, and the like) depending on the nature of the device. For example, and without limitation, the device may be programmable logic hardware, a mainframe computer, server, and personal computer, cloud based program or system, laptop, personal data assistance, cellular telephone, smartphone, or tablet device.

In addition, throughout this specification and the appended claims the term “communicative” as in “communicative pathway,” “communicative coupling,” and in variants such as “communicatively coupled,” is generally used to refer to any engineered arrangement for transferring and/or exchanging information. Examples of communicative pathways include, but are not limited to, electrically conductive pathways (e.g., electrically conductive wires, physiological signal conduction), electromagnetically radiative pathways (e.g., radio waves), or any combination thereof. Examples of communicative couplings include, but are not limited to, electrical couplings, magnetic couplings, radio couplings, or any combination thereof.

It should also be noted that there may be some elements that are used to implement at least part of the embodiments described herein that may be implemented via software that is written in a high-level procedural language such as object-oriented programming. The program code may be written in C, C⁺⁺ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media or a device that is readable by a programmable device. The software program code, when read by the programmable device, configures the programmable device to operate in a new, specific and predefined manner in order to perform at least one of the methods described herein.

Furthermore, at least some of the programs associated with the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

Accordingly, any module, unit, component, server, computer, terminal or device described herein that executes software instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by an application, module, or both. Any such computer storage media may be part of the device or accessible or connectable thereto.

Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be described in the examples herein. Any method, software application or software module herein described may be implemented using computer readable/executable instructions that may be stored or otherwise held by such computer readable media and executed by the one or more processors.

It should also be noted that a description of an embodiment with several components in communication with each other does not imply that all such components are required. On the contrary a variety of optional components are described to illustrate the wide variety of possible embodiments in accordance with the teachings herein.

Further, although process steps, method steps, algorithms or the like may be described (in the disclosure and/or in the claims) in a sequential order, such processes, methods and algorithms may be configured to work in alternate orders. In other words, any sequence or order of steps that may be described does not necessarily indicate a requirement that the steps be performed in that order. The steps of processes described herein may be performed in any order that is practical and provides utility. Further, some steps may be performed simultaneously depending on the situation.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

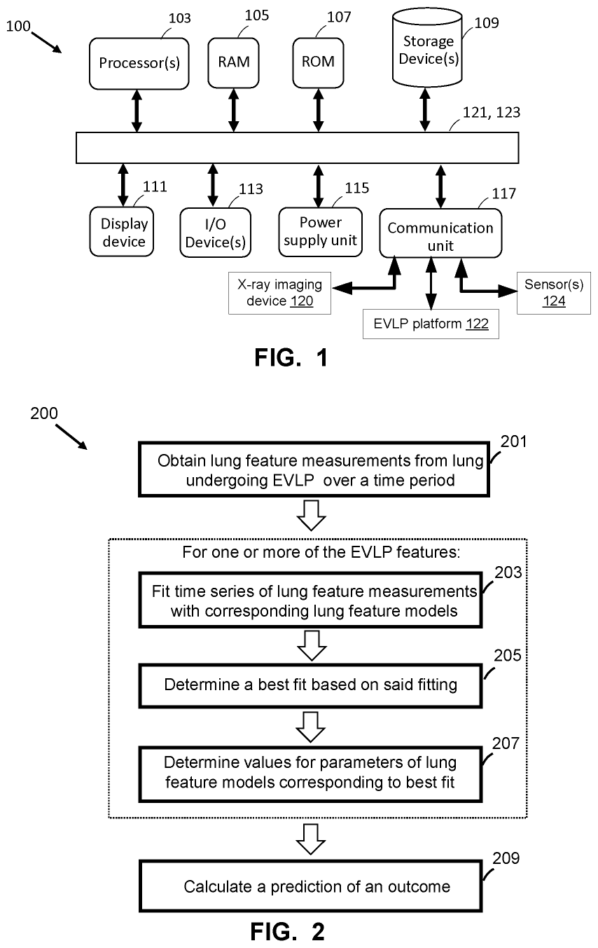

Referring now to FIG. 1, there is shown an example embodiment of an electronic device 100 that may be used for predicting transplant suitability of a donor lung undergoing ex vivo lung perfusion and/or patient outcome following transplant of the donor lung in accordance with the teachings herein. The electronic device 100 may be implemented as a desktop computer, a tablet computer, a mobile device such as a smart phone, or any other suitable device capable of executing software. The electronic device 100 may be used to implement any of the entities, methods, components or services described in the present subject matter.

The electronic device 100 may include one or more processor (“processor(s)”) 103, memory including RAM 105 and ROM 107, one or more storage device(s) 109 (e.g., disk drives, USB keys), a display device 111, input/output (I/O) devices 113 (e.g., a keyboard, at least one pointing device, a microphone, and/or a speaker), a power supply unit 115 and a communication unit 117 that may all send and transmit data over an interconnect 121 (e.g., communication bus and/or data bus) and receive power from a power bus 123. The interconnect 121 may represent any one or more separate physical buses, point to point connections, or both connected by appropriate bridges, adapters, or controllers that allow the various components 103 to 117 to communicate with one another. The interconnect 121, therefore, may include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Components (IEEE) standard 1394 bus, also called “Firewire”.

The processor(s) 103 execute an operating system, and various software programs (also known as software modules), as described below in greater detail. In embodiments where there are two or more processors, these processors may function in parallel and perform certain functions. The processor(s) 103 control the operation of the electronic device 100 and in some embodiments other components of a system described below. The processor(s) 103 may be any suitable processor(s), controller(s) or digital signal processor(s) that can provide sufficient processing power depending on the configuration and operational requirements of the electronic device 100. For example, the processor(s) 103 may include a high-performance processor. Alternatively, in at least one embodiment special-purpose hardwired (non-programmable) circuitry may be used, which may be in the form of, for example, one or more A S ICs, PLDs, FPGAs, etc.

The memory can include the RAM 105, the ROM 107, and one or more storage device(s) 109, which are computer-readable storage media that store software programs having software instructions that implement at least portions of the described embodiments. The RAM 105 provides relatively responsive volatile storage to the processor(s) 103. The ROM 107 is non-volatile storage that stores statis data and program instructions, including computer-executable instructions, for implementing the operating system and software modules (e.g., computer programs), as well as storing any data used by these software modules. The storage device 109, such as a magnetic disk or optical disk, can be provided and coupled to bus 121 for storing information and instructions.

The data may be stored in database or data files, such as for data relating to lungs, donors and/or patients that are assessed using the electronic device 100. The database/data files can be used to store data such as device settings, parameter values, and machine learning models. The database/data files can also store other data required for the operation of the electronic device such as dynamically linked libraries and the like. During operation of the electronic device 100, the software instructions for the operating system, and the software modules, as well as any related data may be retrieved from the non-volatile storage and placed in RAM to facilitate more efficient execution. The memory can also be used for storing temporary variables or other intermediate information during execution of instructions to be executed by processor(s) 103. Other computing structures and architectures may be used as appropriate.

The memory 105, 107 and the storage device(s) 109 are communicatively coupled to the electronic device 100 so that the software instructions of the software programs stored on the memory 105, 107 and/or the storage device(s) 109 can be accessed and executed by the processor(s) 103 of the electronic device 100, which then configures the electronic device 100 to perform one or more of the methods described in the present subject matter. In addition, the data structures and message structures may be stored or transmitted via a data transmission medium, such as a signal on a communications link. Various communications links may be used, such as the Internet, a local area network, a wide area network, or a point-to-point dial-up connection. Thus, computer readable media can include computer-readable storage media (e.g., “non-transitory” media) and computer-readable transmission media.

The software instructions stored in memory 105, 107 can be implemented using any appropriate software development environment or computer language such as high-level program code and/or firmware to configure the processor(s) 103 to carry out actions described above. In some embodiments, such software or firmware may be initially provided to the electronic device 100 by downloading it from a remote system via the communication unit 117. In at least one embodiment, the software program may be provided as a packaged software product, a web-service, an API or any other means of software service.

The display device 111 can be any suitable display that provides visual information depending on the configuration of the electronic device 100. For instance, the display device 111 can be a monitor and the like if the electronic device 100 is a desktop computer. In other cases, the display device 111 can be a display suitable for a laptop, tablet or handheld device such as an LCD-based display and the like. The display device 111 can provide notifications to the user of the electronic device 100. In some cases, the display device 111 may be used to provide one or more GUIs through an Application Programming Interface. A user may then interact with the one or more GUIs for configuring the electronic device 100 to operate in a certain fashion.

The I/O devices 113 allow the user to provide input via an input device, which may be, for example, any combination of a mouse, a keyboard, a trackpad, a thumbwheel, a trackball, voice recognition, a touchscreen and the like depending on the particular implementation of the electronic device 100. The I/O devices 113 also include at least one output device that can be used to output information to the user, which may be, for example, any combination of the display device 111, a printer or a speaker.

For example, one of the input devices may include alphanumeric and other keys, can be coupled to bus 121 for communicating information and command selections to the processor(s) 103. Another type of user input device is a cursor control, such as a mouse, a trackball or cursor direction keys for communicating direction information and command selections to the processor(s) 103 and for controlling cursor movement on the display device 111. For example, the cursor control input device can have two degrees of freedom in two axes, a first axis (e.g., x) and a second axis (e.g., y), that allows the cursor control input device to specify positions in a plane. However, it should be understood other types of input devices allowing for 3 dimensional (x, y and z) cursor movement are also contemplated herein.

The power supply unit 115 can be any suitable power source or power conversion hardware that provides power to the various components of the electronic device 100. The power supply unit 115 may be a power adaptor or a rechargeable battery pack depending on the implementation of the electronic device 100 as is known by those skilled in the art. In some cases, the power supply unit 115 may include a surge protector that is connected to a mains power line and a power converter that is connected to the surge protector (both not shown). The surge protector protects the power supply unit 115 from any voltage or current spikes in the main power line and the power converter converts the power to a lower level that is suitable for use by the various elements of the electronic device 100. In other embodiments, the power supply unit 115 may include other components for providing power or backup power as is known by those skilled in the art. The power supply unit 115 is coupled to the power bus 123 and provides a power signal thereto for providing supply voltage to the other components of the electronic device 100 as needed.

The communication unit 117 enables the electronic device 100 to communicate with other devices via a wired or wireless connection. Accordingly, the communication unit 117 may include network adapters (e.g., network interfaces) for an Internet, Local Area Network (LAN), Ethernet, Firewire, modem or digital subscriber line connection. Alternatively, or in addition thereto, the communication unit 1014 may include a modem and/or a radio that may communicate utilizing CDMA, GSM, GPRS or Bluetooth protocol according to standards such as IEEE 802.11a, 802.11b, 802.11g, or 802.11n.

In at least one embodiment, there may be provided a system comprising the electronic device 100 and an EVLP platform (not shown) that are communicatively coupled to one another. The EVLP platform is known to those skilled in the art.

In at least one embodiment, there may be provided a system comprising the electronic device 100, an x-ray imaging device 120 and an EVLP platform 122 where the electronic device 100 is communicatively coupled to the x-ray imaging device 120 and the EVLP platform 122. The x-ray imaging device 120 is suitable for imaging a donor lung that is contained within the EVLP platform 122. For example, the x-ray imaging device 120 may be, but is not limited to, a DRX-Revolution mobile x-ray system.

In at least one embodiment, there may be provided a system comprising the electronic device 100 and one or more sensors 124 where the electronic device 100 is communicatively coupled to sensor(s) 124. The sensor(s) 124 may be used to obtain data regarding the donor and the lung. For example, the sensor(s) 124 may be used to obtain ventilator data that may be used to measure certain lung parameters such as, but not limited to, compliance and/or airway pressure. In at least one embodiment, the sensor(s) 124 may be used to obtain certain blood flow measurements for the donor's lungs such as, but not limited, to real-time blood gas measurements. In at least one embodiment, the sensor(s) 124 may be used to obtain both ventilator data and blood flow measurements from the donor.

Consistent with certain implementations of the present disclosure, results can be provided in response to the processor(s) 103 executing one or more sequences of one or more software instructions contained in the memory 105. Such software instructions can be read into memory 105 from another computer-readable medium or computer-readable storage medium, such as the ROM 107 and/or the storage device 109. Execution of the sequences of software instructions contained in the memory 105 can cause the processor(s) 103 to perform at least one of the methods/processes described herein. Alternatively hard-wired circuitry can be used in place of or in combination with software instructions to implement the present teachings. Thus, implementations of the present teachings are not limited to any specific combination of hardware circuitry and software.

A computer-program product is also described herein. The computer-program product can be used in conjunction with an electronic device. The computer-program product can include a non-transitory computer-readable storage medium and/or a computer-program mechanism embedded therein. The computer-program product includes program instructions for performing any of the methods described herein.

In an embodiment, the computer-program product may be packaged in software. For example, the computer program product may be available (e.g., for sale, testing, etc.) on the Internet through an online platform (such as a university or hospital website). For example, the computer program product may be available for sale through an online commerce platform.

The term “computer-readable medium” (e.g., data store, data storage, etc.) or “computer-readable storage medium” as used herein refers to any media that participates in providing software instructions to the processor(s) 103 for execution. Such a medium can take many forms, including but not limited to, non-volatile media, volatile media, and transmission media. Examples of non-volatile media can include, but are not limited to, optical, solid state, magnetic disks, such as the storage device 109. Examples of volatile media can include, but are not limited to, dynamic memory, such as memory 105. Examples of transmission media can include, but are not limited to, coaxial cables, copper wire, and fiber optics, including the wires that include bus 121. Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable media, data can be provided as signals on transmission media included in a communications apparatus or system to provide sequences of one or more instructions to the processor(s) 103 of the electronic device 100 for execution. For example, a communication apparatus may include a transceiver having signals that encode software instructions and data. The software instructions and data, when executed by the processor(s) 103, configure the processor(s) 103 to cause the processor(s) 103 to implement one or more of the functions outlined in the disclosure herein. Representative examples of data communications transmission connections can include, e.g., telephone modem connections, wide area networks (WAN), local area networks (LAN), infrared data connections, NFC connections, etc.

It should be appreciated that the methodologies described herein, including flow charts, diagrams and accompanying disclosure can be implemented using the electronic device 100 as a standalone device or on a distributed network of shared computer processing resources such as a cloud computing network.

In at least one embodiment, a computer-implemented method for predicting an outcome for a lung undergoing ex vivo lung perfusion (EVLP) described in accordance with the teachings herein as it relates to use as a donor lung in a recipient post-transplant can employ the use of a processor/device/system as disclosed in the present subject matter. The outcome can be suitability for transplant or patient outcome after transplant. In either case, an electronic device comprising a processor is coupled to a memory storing computer program code to implement one or more of the methods described in the present subject matter. The electronic device 100 is also coupled to memory 105, 107 and/or to storage device(s) 109 to access computer programs and data files including a data database for performing these methods. The electronic device 100 may accept user input from a data input device, such as a keyboard, input data file, or network interface, or another system. The electronic device 100 may provide an output to an output device such as a printer, the display device 111, a network interface, or a data store which may be stored on the storage device(s) 109.

The output device may provide a visual output (e.g., on the display device 111) or output data sent to another electronic device used by a medical professional) including one or more numbers, a graph; a score, etc. to indicate the prediction of transplant suitability of a donor lung undergoing ex vivo lung perfusion and/or patient outcome following transplant of the donor lung. This output may be used by a medical professional, such as a surgeon, to perform one or more actions described herein such as, but not limited to, proceeding with a transplant of the donor lung when a good outcome following transplant is predicted, for example.

As shown in FIG. 2, there is shown a flowchart diagram of an example embodiment of a method 200 for predicting outcomes for a lung undergoing EVLP. At step 201, the method 200 includes obtaining EVLP data for measuring lung features of the lung undergoing ELVP. For example, some the EVLP data may be obtained from the perfusate of the EVLP. In some cases, the EVLP data may be based on perfusate samples. In some cases, the EVLP data may optionally further include real-time or near real-time measurements from the donor lung such as compliance, airway pressure and real-time blood gas measurements. At step 201, the method 200 includes measuring levels (e.g., amplitude, rate, concentration, etc.) for the lung features based on the EVLP data obtained over the time period. For example, levels of biomarkers may be determined from the perfusate samples obtained over the time period. In such cases, the biomarkers can include GM-CSF, IL-10, IL-1β, IL-6, IL-8, sTNFR1, and/or sTREM1.

The EVLP data can be obtained every 15 minutes (e.g., biomarker levels) over a period of time such as, but not limited to, 0-180 minutes of perfusion, for example. The EVLP data, can also be obtained more frequently, for example, on the order of milliseconds, seconds or 1, 2, 3, 4, 5, or 10 minutes from the sample time period depending on the lung feature that is being measured. For example, for physiological parameters (e.g., PO2, PCO2, compliance, airway pressure, blood gas and the like) and biochemical parameters (e.g., pH, electrolytes, glucose and the like), it is possible to have inline, continuous measurements which may be less than 1s. The predetermined period of time can be any time between several milliseconds to 180 minutes depending on the lung feature measurements being made.

At step 203, the method 200 includes, for one or more of the lung features, fitting a time series of the measured values for the lung features (e.g., levels of one or more biomarker parameters) with a corresponding lung feature model. Different mathematical models can be used for fitting the time series of the different lung feature measurements as shown by the examples given in study #2 described below for biomarker parameters. For example, the lung feature models can include, but are not limited to, a linear model, a quadratic model, an exponential model, a 4PL model, or a 5PL model. The equations for the corresponding lung feature model may be obtained from memory 105.

At step 205, the method 200 includes determining a best fit of the parameters of the corresponding lung feature model to the time series that is obtained from the measurements for the lung feature. Various methods may be used to determine the best fit as is known to those skilled in the art.

At step 207, the method 200 includes determining values for the parameters of the corresponding lung feature models after performing the fitting, which may be done using the best fit.

At step 209, the method 200 includes calculating a prediction of an outcome for an individual who receives the lung with a machine learning model. The lung feature model parameters are used as inputs to the machine learning model and the machine learning model output is a prediction of the outcome. In at least one embodiment, various combinations of lung features may be used as inputs to the machine learning model. The outcome can be, but is not limited to, an ICU length of stay or an amount of time of intubation, for example.

In at least one embodiment, the machine learning model may be a univariate logistic regression model, a multivariate logistic regression model, a neural network, a decision tree or ensemble of trees such as random forests and the XGBoost algorithm.

In at least one embodiment, values for features based on lung scores and/or AI-based image processing as described in Applicant's co-pending PCT patent application, entitled “ASSESSMENT OF ex vivo DONOR LUNGS USING LUNG RADIOGRAPHS” that claims priority from US provisional patent application having Ser. No. 63/314,930 filed on Feb. 28, 2022, which is hereby incorporated by reference, may be provided as input to the machine learning model.

The machine learning model may be trained using various known techniques with suitable training datasets, an example of which is described in Study #2 described herein. For example, the XGBoost (Extreme Gradient Boosting) algorithm is a popular machine learning algorithm for supervised learning tasks, such as classification and regression. It is an ensemble learning method that combines multiple decision trees to make more accurate predictions. XGBoost starts by initializing a single decision tree with a root node that contains all of the training samples. The algorithm calculates the gradient (the rate of change) of a loss function with respect to the prediction for each training sample. This is used to determine how much each sample contributes to the overall loss function. The algorithm then tries to find the best split points in the decision tree that will minimize the loss function. It considers all possible split points for each node and chooses the split points that results in the greatest reduction in the loss function. After the split points are found, the algorithm creates a new branch for each split point and continues to recursively grow the tree until a stopping condition is met. The stopping condition may be a maximum depth limit, a minimum number of samples required to create a new node, or a minimum reduction in the loss function. Once the first decision tree is trained, XGBoost creates a new tree that focuses on the samples that the first tree predicted incorrectly. This process is repeated multiple times, with each new tree attempting to correct the errors of the previous trees. Once all the trees are trained, the final prediction is made by combining the predictions of all the trees. The algorithm calculates the weighted average of the predictions, where each tree's prediction is weighted by its performance during training. To prevent overfitting, the XGBoost algorithm uses regularization techniques such as L1 and L2 regularization and pruning to remove nodes that do not contribute to the overall performance of the model.

In at least one embodiment, the output of the machine learning model may include two or more classes. For example, the classes may include three-outcome classifications including: (i) lung unsuitable for transplantation; (ii) EVLP transplant resulting in a time to extubation of >72 hours; and (iii) EVLP transplant resulting in a time to extubation of <72 hours.

In at least one embodiment, the output of the machine learning model may include a probability for each of the classes. For example, in XGBoost, the final probabilities of each class are determined through a combination of the predictions from all of the individual trees in the ensemble. The predicted probabilities are transformed using the logistic function, which maps any value in a range of negative infinity to positive infinity to a value between 0 and 1. This ensures that the predicted probabilities are valid probabilities that sum to 1, and can be interpreted as the likelihood of each class.

In at least one embodiment, the method 200 may additionally include processing certain measurements of the lung features such as processing the levels of the biomarkers to account for circuit dilution prior to the step of fitting the time series of the levels of the biomarkers with the corresponding lung feature models (e.g., biomarker models). Another example of data preprocessing is in embodiments where ventilator data is preprocessed by performing breath segmentation and breath feature extraction to aid in lung physiology analysis (this is described in more detail later).

In at least one embodiment, the method 200 can include using standardized lung feature measurements to correct the levels of the measurements of some of the lung features. For example, Z-score standardization was used to scale the data to have a mean of 0 and a standard deviation of 1. It is a popular method for standardizing continuous data, especially when the data is normally distributed. It is also useful when comparing features with different units or scales.

In at least one embodiment, the method 200 can include using standardized perfusate data to correct the levels of the biomarkers. Perfusate exchange (e.g., removal of old perfusate and the addition of new perfusate) happens during EVLP. The volume of perfusate exchange is recorded and used to calculate perfusate circuit dilution factors for correcting the biomarker levels.

In another aspect, according to at least one embodiment, an electronic device for predicting a lung transplant outcome for an ex vivo lung perfusion (EVLP) is described in the present subject matter. The electronic device can be similar to the electronic device 100 shown in FIG. 1. The electronic device can include one or more user interfaces for receiving user input and EVLP data as well as for providing output indications to the user, a memory and at least one processor that is communicatively coupled to the memory and the one or more user interfaces. The processor(s) can be configured to: determine levels of lung features, optionally biomarkers from EVLP data taken over a time period; for one or more of the lung features, optionally including biomarkers features, fit a time series of the levels of the lung features, optionally include biomarkers, with a corresponding lung feature model, optionally including biomarker lung feature models, which may be done based on a best fit, and determine values for parameters of the corresponding lung feature model, optionally including biomarker models, from the fitting; and calculate a prediction of an outcome for an individual who receives the lung with a machine learning model, wherein the lung feature model parameter, optionally including biomarker model parameters, are used as inputs to the machine learning model and the machine learning model outputs the prediction of the outcome.

In at least one embodiment, a computer program product can include a computer readable memory storing computer executable instructions thereon that when executed by a computing device perform the method steps in the present subject matter, such as the method steps described in FIG. 2.

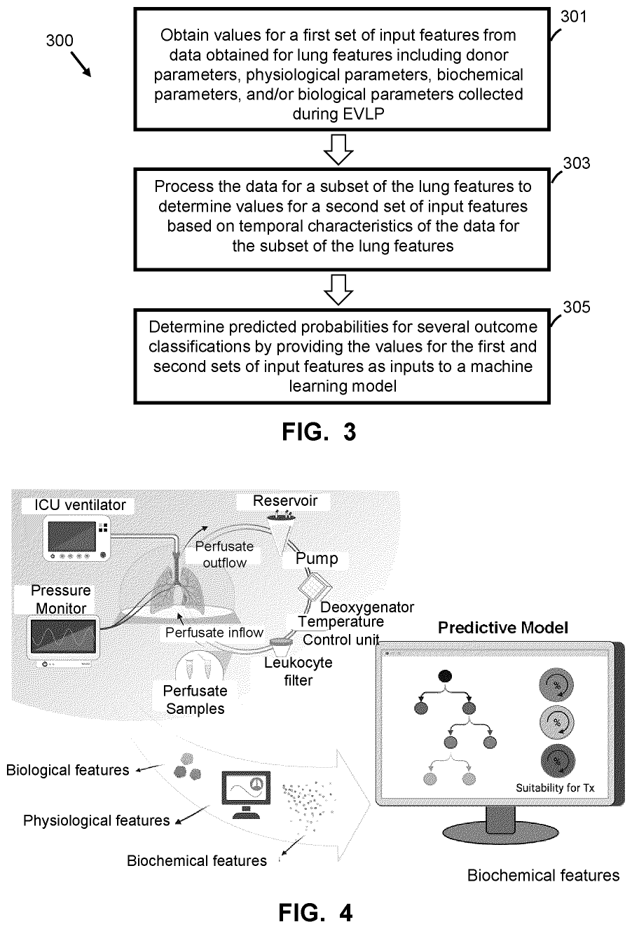

Referring now to FIG. 3, there is shown a flowchart diagram of an example embodiment of a method 300 for predicting outcomes for a lung undergoing EVLP. The method 300 is computer implemented and may be performed by the processor(s) 103, for example.

At step 301, the method 300 includes obtaining values for a first set of features from data obtained for lung features including one or more of donor parameters, one or more recipient parameters, physiological parameters, biochemical parameters, and/or biomarker parameters collected during EVLP.

In at least one embodiment, the donor parameters can include but are not limited to: age; sex; body mass index (BMI); donor type donation-after-brain-death (DBD); donor total lung capacity (TLC) and/or donation-after-cardiac-death (DCD), for example.

In at least one embodiment, the recipient parameters can include but are not limited to one or more recipient physiological features and/or one or more recipient status features. Examples of recipient physiological features include recipient age, recipient sex, and/or recipient BMI, for example. Examples of recipient status include status at assessment, listing, and transplant admission and indication for transplant.

Recipient status is usually assessed at different time points. Recipient status at assessment means the medical status of the patient before being placed onto the waiting list. Recipient status at listing is the medical status of a patient who has been evaluated and has been placed on the waiting list for a suitable donor lung. There are also similar scores in other countries. For example, there is the lung allocation score (LAS), which is used in the US to prioritize lung transplant candidates based on medical urgency and likelihood of success after transplant.

In at least one embodiment, the physiological parameters can include but are not limited to: change in oxygen partial pressure (ΔPO2); change in carbon dioxide partial pressure (ΔPCO2); pH, dynamic compliance; ventilator air flow, static compliance; pulmonary artery (PA) & left atrial (LA) pressure; vascular resistance; airway pressure including peak, mean and plateau; positive end-expiratory pressure (PEEP); edema; perfusate loss; and/or +/−exchange, for example.

In at least one embodiment, the biochemical parameters can include but are not limited to: Ca²⁺; Cl⁻; K⁺; Na⁺; base excess; HCO₃⁻; pH; glucose; and/or lactate, for example.

In at least one embodiment, the biomarker parameters can include but are not limited to: GM-CSF; IL-10; IL-1β; IL-6; IL-8, sTNFR1, and/or sTREM1, for example.

In at least one alternative embodiment, step 301 may also comprise obtaining values for features that are based on lung feature model parameters, such as optionally biomarker model parameters, for one or more lung feature models, such as optionally biomarker models, that are used to model the time series from the measurements of one or more lung features, such as one or more corresponding biomarker levels, as was described with reference to method 200 of FIG. 2.

In at least one additional alternative embodiment, step 301 may also comprise obtaining values for features based on lung scores and/or AI-based image processing as described in Applicant's co-pending PCT patent application entitled “ASSESSMENT OF ex vivo DONOR LUNGS USING LUNG RADIOGRAPHS”.

At optional step 303, the method 300 includes processing the data for a subset of the lung features to determine values for a second set of features based on temporal characteristics (also known as kinetic models) of the data for the subset of the lung features. The temporal characteristics may include one or more statistics such as, but not limited to, a minimum value, a maximum value, a last recorded value and/or a trend (e.g., rate of change), for example, for the data collected for the subset of the parameters. This step may be optional, since in at least one embodiment a Machine Learning (ML) model may effectively extract these features on its own. For example, recurrent neural networks or Transformers can be used to automatically extract these features based on the time-series data.

At step 305, the method 300 includes determining predicted probabilities for several outcome classifications by providing the values for at least one of the first and second sets of lung features as inputs to a ML prediction model.

For example, in at least one embodiment, the ML prediction model can output predicted probabilities for three-outcome classifications including: (i) lung unsuitable for transplantation; (ii) EVLP transplant resulting in a time to extubation of >72 hours; and (iii) EVLP transplant resulting in a time to extubation of <72 hours.

In at least one embodiment, the outcome comprises predicted mechanical ventilation length of time or time to extubation.

In at least one embodiment, the ML prediction model can be implemented using a decision tree algorithm.

For example, in at least one embodiment, the ML prediction model can be implemented using an extreme gradient boosting (XGBoost) machine learning algorithm.

Alternatively, in at least one embodiment, the ML prediction model can be implemented using random forests, support vector machines or a multi-layer perceptron.

The ML prediction model may be trained using various known techniques with suitable training datasets, an example of which is described in Study #1 described herein. For example, the training may use training data from measurements for these features to provide as output a predicted transplant suitability of a donor lung undergoing ex vivo lung perfusion and/or patient outcome following transplant of the donor lung.

In at least one embodiment, an electronic device or a system for predicting a lung transplant outcome for an ex vivo lung perfusion (EVLP) is described in the present subject matter. The electronic device can be similar to the electronic device 100 disclosed in FIG. 1. The electronic device can include one or more user interfaces for receiving user input and providing indication to the user, a memory, and a processor operatively (i.e., communicatively) coupled to the memory and the one or more user interfaces. The processor(s) 103 when executing software instructions may be configured to perform the method 300 described with respect to FIG. 3.

In at least one embodiment, a computer program product can include a computer readable memory storing computer executable instructions thereon that when executed by at least one processor causes the at least one processor to perform the method steps in the present subject matter, such as the steps of method 300 described in FIG. 3.

The embodiments of the present disclosure described herein are intended to be examples only and it is not intended that the applicant's teachings be limited to such embodiments. The present disclosure may be embodied in other specific forms. Alterations, modifications, and variations to the disclosure may be made without departing from the intended scope of the present disclosure. While the systems, devices, and processes disclosed and shown herein may comprise a specific number of elements/components/steps, the systems, devices, and processes may be modified to include additional or fewer of such elements/components/steps. For example, while any of the elements/components/steps disclosed may be referenced as being singular, the embodiments disclosed herein may be modified to include a plurality of such elements/components/steps. Selected features from one or more of the example embodiments described herein in accordance with the teachings herein may be combined to create alternative embodiments that are operable and have utility but are not explicitly described. All values and sub-ranges within disclosed ranges are also disclosed. The subject matter described herein intends to cover and embrace all suitable changes in technology. The entire disclosures of all references recited above are incorporated herein by reference.

### EXAMPLES

### Example 1—Study #1

### Introduction

Precision medicine for isolated organs has been enabled by the development of ex vivo perfusion systems for the lung,¹ liver,² heart,³ kidney,⁴ and pancreas.⁵ For surgeons, these platforms represent a pragmatic approach to assess the suitability of marginal donor organs for transplantation.^{6,7} Ex vivo lung perfusion (EVLP) is an established ex vivo organ system that has been shown to provide a critical relief for patients awaiting transplant through the recovery of donor lungs that would have otherwise been discarded.^{1,8,9} While global lung transplant volumes have increased, they are still significantly outpaced by the number of people added to the waitlist each year—a problem compounded by the recent pandemic.¹⁰ Although EVLP has been shown to be a possible solution to the organ shortage problem,¹¹ the use of EVLP is limited by the lack of standardized definitions of suitable lungs based on the many assessments performed in the ex vivo operating room.^{12,13}

During EVLP, donor lungs are maintained in a normothermic environment, supplied with deoxygenated acellular perfusate solution, and ventilated using an ICU-grade ventilator.^{1,8} At present, lung monitoring includes physiological (i.e., gas exchange, compliance, airway pressure), biochemical (i.e., glucose and lactate levels, pH, acid-base chemistry), imaging (i.e., radiographic images, bronchoscopy), and biological measurements (i.e., inflammatory mediators).^{1,8} Recent research has shown that these assessments are associated with lung injury and patient outcomes;¹⁴⁻¹⁸ however, these studies are limited by the scope of features and do not capture the breadth of potential data derived from EVLP.

The inventors have determined that EVLP is particularly well-suited for ML approaches because the ex vivo data is: (i) restricted to an isolated organ and free of confounding signals from other body systems and (ii) collected longitudinally for several hours. Accordingly, the ex vivo approach enables a wealth of organ-specific data. Furthermore, other data may be used such as donor data and/or recipient data. In this study #1, a machine learning (ML) prediction model was tested to predict transplant outcomes following EVLP and evaluated the impact of the ML prediction model (which may also be referred to as an AI prediction model) on surgical decision-making. However, previous to study #1, evidence that implementation of an AI-guided approach to EVLP decision-making would meaningfully impact organ utilization and post-transplant outcomes had not been demonstrated.

Accordingly, to better understand the predictive nature of EVLP assessments and explore a comprehensive approach to surgical decision-making, the eXtreme Gradient Boosting (XGBoost)¹⁹ ML prediction method was evaluated with over a decade of clinical EVLP data. In this study, the ML prediction model uses donor features and various possible assessments made during EVLP to predict suitable lungs for transplantation and the duration of post-transplant mechanical ventilation. Study #1 was also performed to determine whether or not the ML prediction model may impact clinical decision-making during EVLP.

### Methods

A total of n=601 EVLP cases were used to train (n=504) and test (n=97) an Extreme Gradient Boosting model that was tested for use as the ML prediction model and derived from n=112 features assessed during clinical EVLP. The model was trained to classify: (i) lungs unsuitable for transplantation, or lungs associated with a time to extubation of (ii) ≥72 h or (iii)<72 h post-transplant. To determine the impact of the ML prediction model on decision-making, a subset of clinical cases was independently evaluated by a panel of EVLP specialists. Participants were asked to determine the suitability of the lung for transplant with and without the results from the ML prediction model.

### Findings:

The ML prediction model had an area under the receiver operating characteristic curve (AUROC) of 79% [95% CI: 76-82%] and 75% [95% CI: 75-76%] in the training and test datasets respectively. The ML prediction model performed extremely well in lungs that were unsuitable for transplantation (AUROC: 90% [95% Cl: 86-94%]) and in transplant recipients that were extubated <72 h post-transplant (AUROC: 80% [76-84%]). The use of the ML prediction model greatly increased the recommendation to transplant lungs predicted to have good outcomes [OR=13; 95% Cl:4-45]. This study demonstrates that the inclusion of the ML prediction model can lead to a safe increase in organ transplantation and provides strong rationale for the adoption of ML algorithms during ex vivo organ assessments.

Using the largest clinical dataset available, study #1 demonstrates that a machine-learning approach to EVLP achieves the best reported predictive performance noted to date. The ML prediction model correctly classifies donor lungs across the spectrum of patient outcomes post-transplant. For instance, the ML prediction model may be used to influence surgical decision-making and promote a safe increase in organ utilization rates.

### Methods

### Study Population:

All consecutive clinical EVLP cases performed at Toronto General Hospital (University Health Network, Toronto, ON) from 2008-2020 were considered for model development. Transplant recipient inclusion criteria included adults with end-stage lung disease referred for a first lung transplantation. EVLP exclusion criteria were double lung EVLP assessments that resulted in single lung transplantation.

### Data Collection and Storage:

The EVLP technique has been previously described.¹ Briefly, lung assessments are made hourly and data are derived from an ICU-grade ventilator, pressure monitor, and perfusate samples collected from the EVLP circuit. Additional features were extracted from the donor chart at the time of EVLP. Biochemical and oxygenation data were generated using a blood gas analyzer (RAPIDPoint, Siemens Healthcare, GER). ΔPO2 and ΔPCO2 measurements were calculated as the venous-arterial difference in oxygenation and carbon dioxide levels in perfusate solution. Cytokine measurements (i.e., IL-6, IL-8, IL-10, IL-1β) were completed by ELISA (Protein Simple Inc., CAL, USA and SQI Diagnostics Inc. ON, CAN). Recorded values of perfusate loss and hourly exchanges were used to estimate edema formation during EVLP. A comprehensive list of all assessment features can be found in (Table 2).

### Data Pre-Processinq:

EVLP data was extracted from the Toronto Lung Transplant Database and assessed for completeness. Missing data was obtained using the original source documents and records. For data that was not recorded, an average value was imputed. For each parameter that was assessed hourly during EVLP, the following temporal features were extracted from the data: minimum and maximum values, trend during EVLP, and the last recorded value for a total of four features per parameter. Glucose, lactate, pH, and cytokine features were representative of the fourth hour of EVLP for a total of one feature per parameter. Compliance and cytokine measurements were normalized to lung size using donor total lung capacity. Some data, such as vascular resistance, pulmonary artery (PA) pressure, left atrial (LA) pressure, and airway pressure, may be collected at a higher frequency than hourly.

### MP Prediction Model Development:

In study #1, the ML prediction model was developed using the XGBoost algorithm to predict one of three outcome classifications: (i) lungs unsuitable for transplantation, or EVLP transplants resulting in a time to extubation of (ii) ≥72 h or (iii)<72 h. EVLP cases from 2008-2019 were used to train the model using all donor and EVLP features. k-fold cross validation was used to establish the model parameters in the training data set where k is at least 3 or at least 5. Data arising from EVLP cases conducted from 2019-2020 was used to test the ML prediction model. The predicted probabilities for each EVLP case derived from the ML prediction model was used in the implementation study analysis.

FIG. 4 shows a schematic representation of the ML prediction model according to one example embodiment in which features derived from an ex vivo lung perfusion (EVLP) circuit (top left); and biological, physiological, and biochemical assessments (bottom left) are used as inputs into the ML prediction model (e.g., the XGBoost machine learning algorithm) to predict organ suitability for transplant (bottom right).

### Implementation Analysis:

A subset of EVLP cases were selected for this analysis based on the output of the ML prediction model vs. historical outcome. There were three categories of EVLP cases used in this analysis: controls, low-risk marginal and high-risk transplants. Each case was independently assessed by a review panel that included surgeons, surgical fellows, organ perfusion specialists, and EVLP assistants. Each EVLP case was de-identified and presented alongside donor and recipient information. For cases that were declined, the details of the intended recipient were used. The study cases were randomly selected from the EVLP cohort based on the following categories: (i) confirmatory, (ii) utilization, and (iii) outcome improvement. For the ‘confirmatory’ category of lungs, six cases where the historical outcome matched the ML prediction model outputs (i.e., extubated <72 h and unsuitable for transplant) were selected. A total of 12 cases that were historically declined for transplant, but the ML prediction model predicted that the lungs were likely to produce a good transplant outcome were selected to represent the ‘utilization’ category. Finally, five lungs where the ML prediction model correctly predicted the need for prolonged ventilation were chosen in the ‘outcome improvement’ group. Respondents were asked to determine the suitability of the lung for transplant (yes or no) based on standard EVLP evaluation parameters alone and their assessment of the organ on a scale from 0-10. The ML prediction model results were then revealed and respondents were asked to re-answer the transplant suitability and lung assessment questions.

### Statistical Methods:

Demographics were analyzed using descriptive statistics. Chi-squared or Fisher's exact test was used to determine patient factors associated with clinical outcomes. Kruskal-Wallis, ANOVA, and Mann-Whitney U tests were used to analyze differences in biomarker levels and clinical outcomes. Multiple comparisons were adjusted using Dunn's correction. The area under the receiver operating characteristic (AUROC) curve was used to assess the predictive performance of the ML prediction model with the null hypothesis that predictive performance was 50%. A random effects logistic regression model was fit to the transplant decision and lung assessment data from the retrospective case review to determine the impact of the ML prediction model on the transplant decision and lung assessment score. All analyses were conducted using Stata (StataCorp, TX, USA), GraphPad (GraphPad Software, CA, USA), SPSS Statistics (IBM Corp, NY, USA), Python Programming Language (Python Software Foundation, DE, USA), or R statistics software.

### Results

### EVLP Cohort Characteristics

From 2008 to 2020, there were a total of 601 eligible clinical EVLP cases that were included in the development and validation of the ML prediction model. There were 504 EVLP cases performed from 2008 to November 2019 that were used as a development dataset; EVLP cases conducted between December 2019 and December 2020 (n=97) represented a validation cohort for the ML prediction model (Table 1). There were no significant differences in the donor characteristics or EVLP outcome between the two cohorts (Table 1). However, better post-transplant outcomes were observed more frequently in the validation cohort (Table 1). Of all donor lungs evaluated on EVLP, 40% resulted in transplantation and extubation in less than 72 h post-transplant, 23% of EVLP cases were associated with prolonged ventilation, and 37% of lungs were deemed unsuitable for transplant.

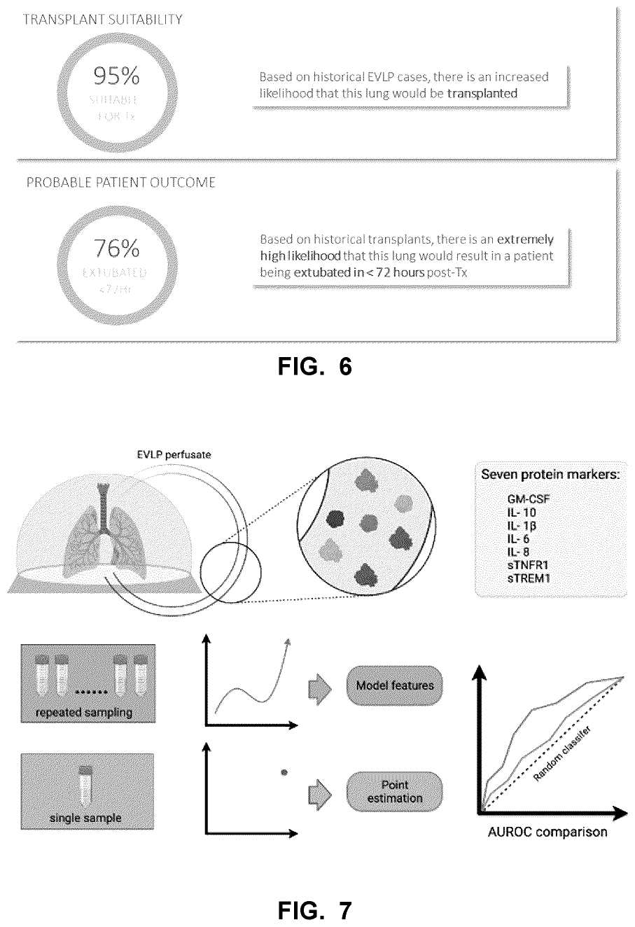

FIG. 6 includes ML prediction results that show the likelihood that the lung in the EVLP is suitable for transplant (top panel) as well as the probability that, if transplanted, a recipient may be extubated in less than 72 hours post-transplant (bottom panel).

TABLE 1
Clinical EVLP case characteristics for
the ML prediction model development

| | Training | Test | |
|---|---|---|---|
| | Dataset | Dataset | p-value |
| Date Range | 2008-2019 | 2019-2020 | — |
| Number of Cases | 504 | 97 | — |
| Mean Age (SD) - Years | 45 (±17) | 48 (±16) | 0.14 |
| Male Sex (%) | 328 (65%) | 62 (64%) | 0.75 |
| Mean BMI (SD) | 27.3 (±6.5) | 27.3 (±6.0) | 0.92 |
| Donor Type DBD (%) | 259 (51%) | 48 (49%) | 0.66 |

EVLP Outcome

| | | | |
|---|---|---|---|
| Transplanted (%) | 313 (62%) | 64 (66%) | 0.47 |
| Declined (%) | 191 (38%) | 33 (34%) | |

Transplant Outcome

| | | | |
|---|---|---|---|
| Extubated <72 h (%) | 190 (38%) | 48 (49%) | 0.07 |
| PGD 3 at 72 h (%) | 57 (11%) | 4 (4%) | 0.02 |
| Median ICU LOS [IQR] - Days | 4 [2-10] | 4 [2-6] | 0.08 |

Legend: SD = standard deviation; BMI = body mass index; DBD = donation after brain death; EVLP = ex vivo lung perfusion; PGD = primary graft dysfunction; ICU = intensive care unit; LOS = length of stay; IQR = interquartile range. Statistics: Mann-Whitney test for age, BMI and ICU LOS; Chi-square test for sex, donor type, EVLP outcome, PGD3, and time to extubation.

### ML Prediction Model Development and Performance

There were 31 parameters arising from donor information, physiological, biochemical, and biological assessments collected for each EVLP case (see Table 2). In order to capture the longitudinal nature of EVLP, additional features for temporal parameters that were assessed hourly were extracted including: the trend, minimum, maximum, and last recorded value for a total of 91 features that were used as inputs for the ML prediction model.

The ML prediction model was developed using the XGBoost algorithm with three endpoints for model classification: (i) donor lungs on EVLP deemed unsuitable for transplantation and, EVLP cases that resulted in transplantation with recipients who were extubated in (ii) less than or (iii) more than 72 h post-transplant. The development cohort was randomly partitioned 80:20 for training and testing, and 5-fold cross-validation was performed on the development dataset. The validation cohort was then used as an additional test dataset for the ML prediction model.

TABLE 2
All EVLP features used in the ML prediction model

| Donor | Physiological | Biochemical | Biomarker |
|---|---|---|---|
| Age | Δ PO2 | Ca²⁺ | IL-10 |
| Sex | Δ PCO2 | Cl⁻ | IL-1β |
| BMI | Dynamic Compliance | K⁺ | IL-6 |
| Type | Static Compliance | Na⁺ | IL-8 |
| (DBD vs DCD) | | | |
| | PA & LA Pressure | Base Excess | |
| | Vascular Resistance | HCO₃⁻ | |
| | Airway Pressure | pH | |
| | (Peak, Mean, Plateau) | | |
| | PEEP | Glucose | |
| | Edema | Lactate | |
| | (Perfusate loss, +/−exchange) | | |

Legend:
ΔPO2 = change in oxygen partial pressure;
ΔPCO2 = change in carbon dioxide partial pressure;
LA = left atrial;
PA = pulmonary artery;
IL-8 = interleukin-8;
IL-6 = interleukin-6;
IL-10 = interleukin-10;
IL-1β = interleukin-1beta;
BMI = body mass index;
DBD = donation after brain death;
DCD = donation after cardiac death;
PEEP = positive end-expiratory pressure

TABLE 3
Confusion matrix for the ML prediction model in the Training and Test datasets

| | | Predicted Outcome | | | |
|---|---|---|---|---|---|
| | | Extubated <72 h | Extubated >72 h | Unsuitable for | |
| | | Post-Transplant | Post-Transplant | Transplant | Total |
| Actual Outcome | Extubated <72 h | 167 | 38 | 33 | 238 |
| | Post-Transplant | | | | |
| | Extubated >72 h | 44 | 69 | 26 | 139 |
| | Post-Transplant | | | | |
| | Unsuitable for | 18 | 18 | 188 | 224 |
| | Transplant | | | | |
| | Total | 229 | 125 | 247 | |

The AUROC for the overall ML prediction model was 79±3% and 75±4% in the training and test sets respectively (Table 4). Importantly, the ML prediction model performed extremely well in donor lungs on EVLP that resulted in a time to extubation less than 72 h (AUROC: 80±4% (training), 76±6% (test)) and in lungs that were unsuitable for transplantation (AUROC: 90±4% (training), 88±4% (test)). The prediction of prolonged extubation in transplant recipients was modest (AUROC: 67±6% (training), 62±9% (test)) (Table 4 However, the precision of the model to identify injured lungs (i.e., unsuitable or extubated >72 h) was very good at 70%. Notably, model precision for non-injured lungs (i.e., extubated <72 h) was similar at 73%. Furthermore, the area under the precision-recall curve (AUPRC) showed a marked improvement of the ML prediction model to predict the desired outcome compared to the baseline AUPRC: 67±6% (training) and 75±8% (test) vs. 40% for patients with short ventilation times, 40±7% (training) and 31±11% (test) vs. 23% for prolonged ventilation post-transplant, and 86±5% (training) and 81±7% (test) vs. 37% in lungs deemed unsuitable for transplant.

TABLE 4
Prediction of EVLP and Tx outcomes
using the ML prediction model

| | Overall | Extubated | Extubated | Unsuitable |
|---|---|---|---|---|
| AUROC (SD) | Performance | <72 h | >72 h | for Tx |
| Training Dataset | 79 ± 3% | 80 ± 4% | 67 ± 6% | 90 ± 4% |
| Test Dataset | 75 ± 4% | 76 ± 6% | 62 ± 9% | 88 ± 4% |
| p-value (train vs test) | p = 0.50 | p = 0.48 | p = 0.49 | p = 0.48 |

Legend: AUROC = area under receiver operating characteristic curve; AUPRC = area under the precision recall curve; SD = standard deviation;
Tx = transplantation.

One characteristic of the XGBoost algorithm is the ability to determine the relative weighting of the input variables. Only Donor Type and PEEP had importance values of 0 and were therefore not required by the ML prediction model for outcome prediction whereas the other input features were used by the ML prediction model. These findings are aligned with observations that donor type is not an important variable after EVLP and that PEEP is constant and unlikely to have predictive value. Interestingly, with the ML prediction model, it was observed that a unique mix of the donor and EVLP parameters that were driving the prediction of each clinical endpoint (Table 5). For lungs that were unsuitable for transplantation, it was determined that physiological parameters (i.e., compliance, oxygenation, airway pressure) were the driving model features (Table 5). Comparatively, transplanted lungs with recipients that had a reduced need for ventilator support were predicted by physiological and biochemical features (Table 5). Notably, Ca²⁺ and IL-8 levels were important features of lungs with good outcomes (Table 5).

TABLE 5
Top 10 ranked EVLP features in the ML prediction model by endpoint

| | Overall Model | Extubated <72 h | Extubated >72 h | Unsuitable for |
|---|---|---|---|---|
| | Performance | Post-Transplant | Post-Transplant | Transplant |
| 1 | Δ PO2 | Static Compliance | Static Compliance | Δ PO2 |
| 2 | Static Compliance | Δ PO2 | Edema | Static Compliance |
| 3 | Peak Airway | Ca²⁺ | Dynamic | Peak Airway |
| | Pressure | | Compliance | Pressure |
| 4 | Dynamic | Base Excess | Peak Airway | Dynamic |
| | Compliance | | Pressure | Compliance |
| 5 | Base Excess | Dynamic | Plateau Pressure | Edema |
| | | Compliance | | |
| 6 | Edema | Edema | IL-8 | Base Excess |
| 7 | Plateau Pressure | Peak Airway | K⁺ | Plateau Pressure |
| | | Pressure | | |
| 8 | Ca²⁺ | Na⁺ | Δ PCO2 | Mean Airway |
| | | | | Pressure |
| 9 | Mean Pressure | pH | LA Pressure | pH |
| 10 | pH | IL-8 | Na⁺ | Ca²⁺ |

Legend:
ΔPO2 = change in oxygen partial pressure;
ΔPCO2 = change in carbon dioxide partial pressure;
LA = left atrial;
IL-8 = interleukin-8

### Implementation Analysis

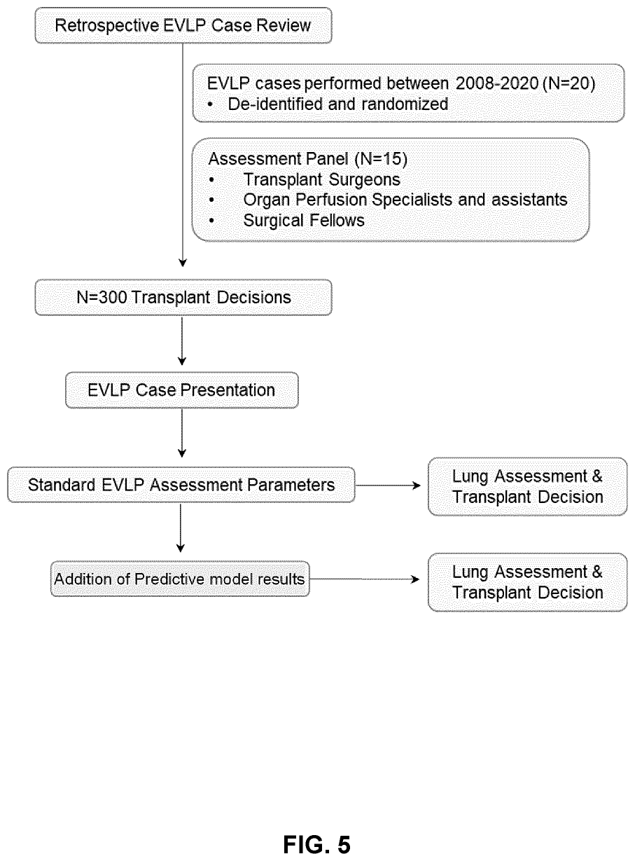

It was also investigated whether or not the results of the ML prediction model may have a meaningful impact on surgical decision-making during EVLP. To evaluate the effect of the ML prediction model, a retrospective case review of 20 EVLP cases was conducted with a panel of 15 participants comprising: surgeons, surgical fellows, organ perfusion specialists, and EVLP assistants (FIG. 5). In particular, FIG. 5 shows a schematic for retrospective EVLP case review with ML prediction model according to an example. Taken together, there were 300 individual transplant decisions from the 20 study cases. A summary of the donor and recipient characteristics are provided in Table 6.

TABLE 6
Intended recipient and donor characteristics for
ML prediction model assessment Study Cases

| Date Range | 2008-2020 | |
|---|---|---|
| Number of Cases | 20 | |
| Mean Age (SD) - Years | | |
| Donor | 45 | (±16) |
| Recipient | 57 | (±12) |
| Male Sex (%) | | |
| Donor | 13 | (65%) |
| Recipient | 12 | (60%) |
| Donor Type DBD (%) | 10 | (50%) |
| Recipient Status (%) | | |
| 1 | 6 | (30%) |
| 2 | 10 | (50%) |
| 3 | 4 | (20%) |
| Recipient Disease (%) | | |
| Emphysema/COPD | 6 | (30%) |
| Cystic Fibrosis | 2 | (10%) |
| PF/ILD/UIP/NSIP | 10 | (50%) |
| Other | 2 | (10%) |
| EVLP Outcome | | |
| Transplanted (%) | 8 | (40%) |
| Declined (%) | 12 | (60%) |
| Transplant Outcome | | |
| Extubated <72 h (%) | 3 | (38%) |
| PGD 3 at 72 h (%) | 2 | (25%) |
| ICU LOS [IQR] - Days | 5 | [3-7] |

Legend: SD = standard deviation; DBD = donation after brain death; EVLP = ex vivo lung perfusion; PGD = primary graft dysfunction; ICU = intensive care unit; LOS = length of stay; IQR = interquartile range.

Overall, it was observed that the use of the ML prediction model may result in a net increase of 3% in transplant volume and, the effect represented a balanced trade off between an increase in transplant decisions for lungs more likely to produce good outcomes and a decrease in those that were unsuitable for transplant. For the EVLP lungs in the ‘confirmatory’ group, the use of the ML prediction model may have led to an increase of 7% in organ utilization for lungs with good outcomes and a decrease in utilization by 5% for lungs unsuitable for transplantation (Table 7). Interestingly, a net decrease of 13% was observed for the utilization lungs that resulted in the need for prolonged ventilation with no change in the lung assessment score (Table 7). For lungs that were historically declined but predicted to be suitable by the ML prediction model, there was a 13% increase in organ utilization when the ML prediction model results were available (Table 7). Using a mixed effects logistic regression approach to model the impact of transplant decisions and lung assessment scores, a clinically meaningful impact of the ML prediction model on surgical decision-making was observed. Notably, for lungs that were suitable for transplantation, the ML prediction model resulted in an odd ratio of 13 [95% Cl: 4 to 45] in transplant decisions and an improvement of 9-5% [95% Cl: 4 to 15-1%] in lung suitability assessments (Table 8).

TABLE 7
Summary of the impact of the ML prediction
model on clinical decision-making

| | ML prediction | No. | ML prediction vs. SOC | |
|---|---|---|---|---|
| Historical | model Predicted | Deci- | ΔEVLP | ΔLung |
| Outcome | Outcome | sions | Utilization | Impression |
| Extubated <72 h | Extubated <72 h | 45 | +7% | +10% |
| Post-Transplant | Post-Transplant | | | |
| Unsuitable for | Unsuitable for | 45 | −5% | −10% |
| Transplant | Transplant | | | |
| Extubated >72 h | Extubated >72 h | 75 | −13% | 0 |
| Post-Transplant | Post-Transplant | | | |
| Unsuitable for | Extubated <72 h | 135 | +13% | +5% |
| Transplant | Post-Transplant | | | |

Legend: SOC = standard of care; EVLP = ex vivo lung perfusion.

TABLE 8
Summary of the impact of the ML prediction model on clinical decision-making

| | Transplant Decision | | Lung Assessment Score | |
|---|---|---|---|---|
| | OR [95% CI] | | Δ % [95% CI] | |
| Suitable Donor Lungs | 13 | [95% CI: 4 to 45] | +9.5% | [95% CI: 4 to 15.1%] |
| Unsuitable Donor Lungs | 0.4 | [95% CI: 0.16 to 0.98] | −3.1% | [95% CI: −7.5 to 1.4%] |

Legend: OR = odds ratio; CI = confidence interval

### Discussion

In study #1, it was observed that an ML approach to organ assessment accurately predicts EVLP and post-transplant outcomes. The ML prediction model was developed using the largest collection of clinical EVLP cases to date. The ML prediction model performed extremely well for the prediction of three possible outcomes following EVLP with an AUROC of 79% and 75% in the training and test datasets. It was shown that the ML prediction model represents a surgical decision-aid that may lead to a safe increase in transplant volume at our institution.

Previous studies have shown the predictive nature of the various biomarkers during EVLP.^{14-18,20-25} A study by DiNardo et al. demonstrated that physiological and biochemical features were associated with the decision to transplant.¹⁴ In addition, numerous other studies have highlighted the predictive role of inflammatory cytokines, including IL-6, IL-8, IL-10, and IL-1β for the assessment of lung injury.¹⁵⁻¹⁸ As such, the approach taken in the present study attempted to encapsulate all of the available data and research conducted to date towards the development of a comprehensive EVLP assessment model using ML.

Historically, most studies on EVLP biomarker studies have focused on dichotomous endpoints and therefore fail to adequately represent the spectrum of outcomes following EVLP. A unique feature of the ML prediction model is the reporting of the likelihood of all of the possible clinical outcomes following EVLP. This provides surgeons with a more comprehensive view of the lung on EVLP and what recipient outcome is likely to occur post-transplant. Notably, the ML prediction model showed excellent performance in lungs that were unsuitable for transplant and in those that were more likely to result in a short time to extubation post-transplant. However, modest ML prediction model performance was observed in lungs that were associated with prolonged time to extubation which may be due to recipient factors and or a continual improvement in post-operative care.²⁶ Given that the use of intraoperative extracorporeal membrane oxygenation has increased, many marginal lungs on EVLP may be safely guided to good outcomes during and post-transplant.²⁷

The ML prediction model described herein does not include recipient characteristics as part of the predictive input features. The exclusion of recipient details was purposeful and due primarily to the objective of deriving a model that may predict outcome in any recipient, irrespective of their condition or status. Furthermore, it is expected that as the field of ex vivo organ perfusion grows, there will be targeted therapies and regenerative strategies to improve the function of the organ.¹¹ Thus, the ML prediction model appears to be well-suited to meet this future state by focusing on the outcome of the organ and will be able to gauge the impact of any future intervention on a donor lung, thereby aiding to ensure that all donor lungs are well conditioned prior to transplant. As well, the ML prediction model enables the evaluation of the donor lung in isolation, yet the final decision to transplant resides with the surgeon who takes all relevant recipient features into account. Accordingly, after the prediction is made the donor lung predicated as being likely suitable for transplant may be subsequently transplanted into the patient (i.e., the recipient).

Detailed analysis of the ML prediction model revealed a different mix of assessment parameters were driving the various endpoint classifications. While this finding was not unexpected, it was extremely interesting to note the relative importance of various features in relation to lung suitability and patient outcomes. Of note, certain biological and biochemical biomarkers were highly ranked for the prediction of post-transplant outcome. In particular, it appears that acid-base chemistry may be useful in determining patient outcomes. Features such as pH and base excess are biomarkers of metabolic and respiratory acidosis in respirology;²⁸ however, the identification and weighting of these markers in EVLP by the ML prediction model further underscore the value of an AI-based approach to ex vivo assessments.

One of the findings in study #1 was the real-world evaluation of the use of the ML prediction model on surgical decision-making. While there have been reports of predictive ML algorithms in thoracic surgery,²⁹ this is the first such study to show that the use of an AI-based decision-aid during EVLP would change lung transplant decisions. The results of this study suggest that the impact of ML on transplantation rates could dramatic and that an overall increase in transplant activity is plausible. Importantly, effects of ML were different based on the phenotype of a donor lung and the subsequent post-transplant outcome. For lungs that were associated with poor outcomes there was a large decrease in the tendency to transplant which was offset by an even larger increase in lungs that were historically declined, but would have been transplanted had the ML prediction model results been available at the time EVLP. Thus, it is believed that these findings suggest that overall donor lung utilization rates will safely increase with the ML prediction model.

### Example 2—Study #2

### Introduction

As previously noted, Ex vivo lung perfusion (EVLP) is a promising technique to assess donor lung quality and the suitability for transplantation³⁰. As an organ assessment and reconditioning platform, EVLP provides clinicians with more confidence to transplant marginal donor lungs, leading to safe expansion of the donor pool^{31,32}. During EVLP, donor lungs are perfused, stabilized, and maintained at normothermic temperature which enables the precise evaluation of physiological and biochemical parameters to support transplant decisions^{33,34}. Specifically, the circulating perfusate serves as a key source of lung biomarkers, allowing for the study of quantitative changes in important biomarkers in the EVLP circuit and the establishment of dynamic biomarker profiles^{35,38}. One approach to studying perfusate-derived biomarkers involves hourly sampling of perfusate at predefined hourly time points to predict lung transplant outcomes. This approach helps to understand the biomarker concentrations but only provides limited information on dynamic biomarker behaviour during EVLP. It has been shown that EVLP perfusate-derived biomarkers present diagnostic and predictive values for supporting clinical decision during lung transplant; however, there is still a lack of knowledge of the biomarker kinetic profiles that accurately reflect the biomarker behaviour over time. To address this current lack of knowledge in the field, study #2 has been performed to model the kinetic behaviour of time-series biomarker data using various mathematical models. The study tested examined whether the features extracted from best-fit kinetic models may more accurately reflect the biomarker behaviour and therefore improve prediction performance for lung transplant outcomes was further investigated.

A number of previous studies have demonstrated the predictive value of EVLP perfusate-derived protein biomarkers. For example, perfusate concentration of interleukin-8 (IL-8) measured at 4 h of EVLP was predictive of primary graft dysfunction grade 3 (PDG3)³⁹; IL-8 and IL-1β concentrations measured hourly were also used to effectively predict the final EVLP outcome⁴⁰. Moreover, Toronto Lung Score (TLS2), a 2-plex inflammation score established by combining IL-6 and IL-8 levels from hourly perfusate samples, also presented predictive values of PGD3, transplant decision, and recipient outcomes⁴¹. For other markers such as granulocyte-macrophage colony-stimulating factor (GM-CSF), a well-known marker that drives immune function in lungs⁴², has not been widely investigated in EVLP perfusate. Soluble tumour necrosis factor receptor 1 (sTNFR1) was shown to associate with lung macrophage activity⁴³ proinflammatory state³⁶, lung function impairment in obesity⁴⁴, and different lung donor types⁴⁵. On the other hand, soluble triggering receptor expressed on myeloid cells 1 (sTREM1) in bronchoalveolar lavage was reported as a diagnostic biomarker of bacterial lung infection in intensive care unit (ICU) patients⁴⁶ and has been used to predict mortality in hospitalized patients⁴⁷. However, these markers have not been extensively studied in EVLP perfusate.

FIG. 7 shows a schematic of the study overview for study #2. This study describes the kinetic profiles of seven biomarkers (GM-CSF, IL-10, IL-1β, IL-6, IL-8, sTNFR1, and sTREM1) found in EVLP perfusate. The mathematical models used to study biomarker kinetics to determine kinetic model features and the diagnostic and predictive value of the kinetic model features were then compared to standard hourly collection as shown in FIG. 7.

With the data in this particular study, it was found that a linear model best described GM-CSF (r²=0.9156), sTNFR1 (r²=0.9453), sTREM1 (r²=0.9608), whereas, IL-10 (r²=0.9302), IL-1β (r²=0.9198), IL-6 (r²=0.9515), and IL-8 (r²=0.9808) were best described by an exponential growth curve. Features derived from best-fit models were found to be predictive for ICU length-of-stay (<3 days). Combining kinetic model features with hourly data appeared to result in significantly improved diagnostic performance for IL-10 (AUROC=81% [61-99%]), GM-CSF (AUROC=79% [57-99%]), and IL-8 (AUROC=72% [50-94%]). Accordingly, study #2 showed that features derived from kinetic models that accurately reflect protein biomarker time behaviour can improve the prediction of EVLP outcomes.

### Methods

### Ex Vivo Lung Perfusion and Sampling Protocol:

The technique of the Toronto EVLP protocol has been previously described. A total of 45 clinical EVLP cases conducted at the Toronto General Hospital between 2017 to 2019 were included in this study. For each case, EVLP perfusate was sampled every 15 minutes after the start of perfusion until 180 minutes or to the end of EVLP. Perfusate samples were tested immediately or snap-frozen in liquid nitrogen and stored at −80° C. for later analysis.

### Protein Biomarker Measurements:

Seven protein biomarkers were studied: granulocyte-macrophage colony-stimulating factor (GM-CSF), interleukin (IL)-10, IL-1β, IL-6, IL-8, soluble tumour necrosis factor receptor 1 (sTNFR1), and soluble triggering receptor expressed on myeloid cells 1 (sTREM1). All biomarkers were measured in perfusate samples using ELISA-based ELLA Platform (Protein Simple, San Jose, CA, USA) following manufacturer's instructions. Protein concentrations were reported in pg/mL for all biomarkers.

### Circuit Dilution Calculation:

The dilution correction was calculated based on the volume of STEEN removal and addition during EVLP. The STEEN exchange data were recorded by clinical perfusionists and used to calculate dilution factors, which were used to correct all biomarker levels at each corresponding time point.

### Kinetic Models:

Five mathematical models were constructed using R programming to fit time series protein biomarker data for 45 cases: linear, quadratic, exponential, four-parameter logistic (4PL) regression and five-parameter logistic (5PL) regression models. The median goodness of fit values derived from the five models were compared for each biomarker to determine the best-fit model. Any model that has the ability to describe a timeseries relationship may potentially be used to describe the behaviour of the biomarkers. For example, logistic growth models or logarithmic models may be used in some embodiments for certain biomarker models.

For example, the equations of the biomarker models which were studied are:

$$Linear model : y = mx + b$$

$$Quadratic model : y = {ax}^{2} + bx + c$$

$$Exponential model : y = y 0 \times {e}^{kx} or ln ( y ) = mx + b$$

$$4 PL model : y = d + \frac{a - d}{1 + {( \frac{x}{c} )}^{b}}$$

$$5 PL model : y = d + \frac{a - d}{{[ 1 + {( \frac{x}{c} )}^{b} ]}^{s}}$$

### Time Points Random Selection & Model Comparison:

For each biomarker, random selection of time points (n=2-6) was repeated for 11 times to establish comparable models. Model features were compared using the paired t-test. For the linear model, the rate of change value (m) was compared. For the quadratic model, the coefficients of x² (a) and x (b) were compared. For the exponential model, the starting value (y0) and rate constant (k) were compared. For 4PL and 5PL models, the hill slope values (b) were compared.

### Model Features & Recipient Outcome Prediction:

The model features were then used to predict recipient intensive care unit (ICU) length of stay using the area under the receiver operating characteristic curve (AUROC). For example, for the linear model, rate of change (m) and y-intercept (b) were used as model features for prediction. For the exponential model, Y0, k, and the instantaneous rate of change at point estimate were used as model features. ICU length of stay was predicted as binary classification using 3 days as cut-off (total n=22; 11 vs. 11). Model features derived from best-fit models were used individually as univariate features and combined to build a multiple logistic regression model to predict recipient ICU length of stay (<3 days). The point estimation biomarker data (uncorrected for dilution) was used as univariate feature to predict recipient ICU length of stay, which was compared with prediction performance of model features. Only bilateral transplant cases were included for prediction analysis. EVLP cases with poor goodness of fit values (R²<0.5) or missing values were excluded for prediction.

### Statistical Analysis:

Model fit results from pre- and post-dilution correction models were compared using Wilcoxon signed-ranks tests. The predictive power of each predictive feature was assessed using the area under the receiver operating characteristic curve (AUROC). P-values associated with AUROC values were based on Mann-Whitney U statistics⁴⁸ and computed using the “verification” package in R. The Shapiro-Wilk test was used to test if variables follow normal distribution.

### Results

### Protein Biomarkers are Uniquely Described by Different Kinetic Models:

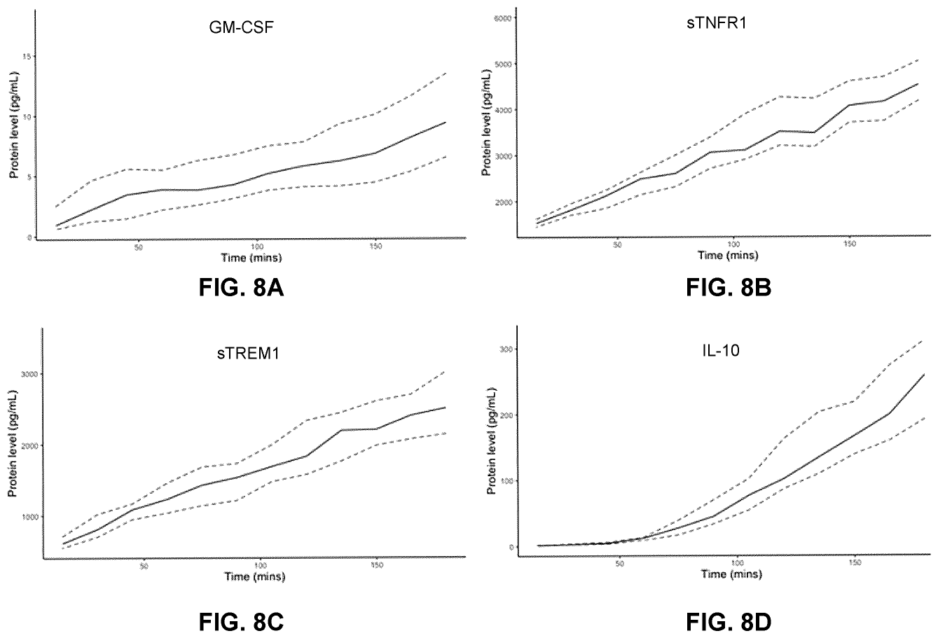

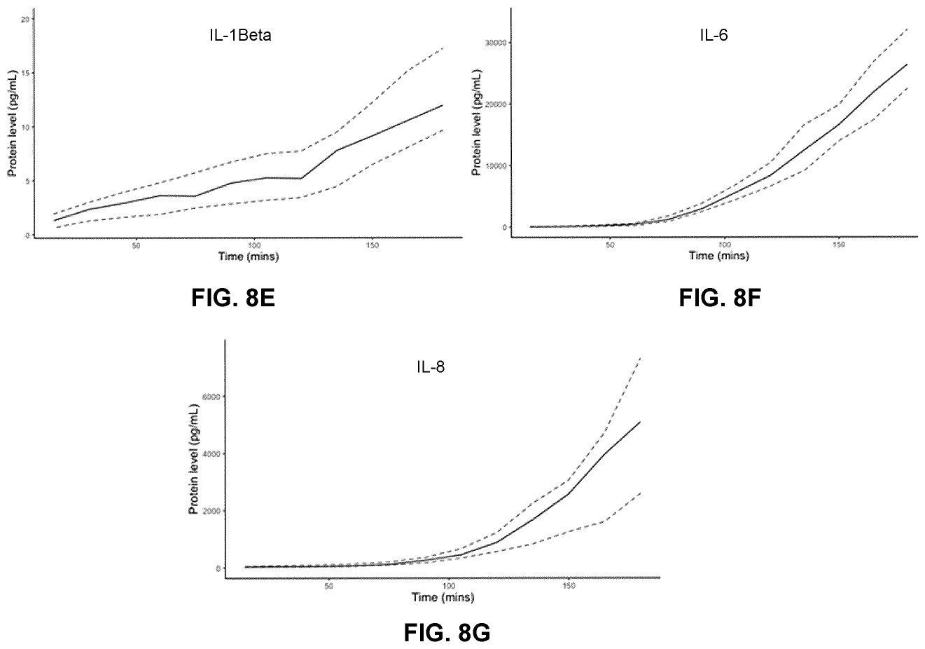

FIGS. 8A-8G shows a time series of the biomarker concentrations (median±95% CI). Y-axes represent biomarker concentrations in pg/ml whereas x-axes represent EVLP duration in minutes. Each panel represent different biomarkers as follow: (A) GM-CSF; (B) sTNFR1; (C) sTREM1; (D) IL-10; (E) IL-1β; (F) IL-6; and (G) IL-8.

Repeated sampling established the time-series data for the seven biomarkers (FIGS. 8A-8G). Goodness of fit values of five kinetic models for all biomarkers are summarized in Table 9. The linear model can be used to best-describe GM-CSF (r2=0.9156), sTNFR1 (r2=0.9453), and sTREM1 (r2=0.9608), whereas IL-10 (r2=0.9302), IL-1β (r2=0.9198), IL-6 (r2=0.9515), and IL-8 (r2=0.9808) were best described by an exponential growth model.

TABLE 9
Median R² values of five mathematical models for seven EVLP biomarkers

| Model | GM-CSF | IL-10 | IL-1β | IL-6 | IL-8 | sTNFR1 | sTREM1 |
|---|---|---|---|---|---|---|---|
| Linear Model | 0.9157 | 0.9298 | 0.8949 | 0.8582 | 0.7794 | 0.9453 | 0.9608 |
| Quadratic Model | 0.9619 | 0.9845 | 0.9665 | 0.9925 | 0.9847 | 0.9518 | 0.9703 |
| Exponential Model | 0.8402 | 0.9302 | 0.9198 | 0.9515 | 0.9808 | 0.9183 | 0.9245 |
| 4PL Model | 0.9602 | 0.9965 | 0.9670 | 0.9960 | 0.9974 | 0.9551 | 0.9653 |
| 5PL Model | 0.9772 | 0.9973 | 0.9772 | 0.9962 | 0.9981 | 0.9607 | 0.9663 |

Note:
all p-values <0.001

### Dilution Correction Significantly Improved Model Fit

Mean R² values from pre- and post-dilution correction model fits are shown in Table 10. Dilution correction significantly increased R² values for improved model fit [p<0.05 (IL-8, GM-CSF); p<0.001 (IL-1β, IL-10, IL-6, sTNFR1, sTREM1)]. It should be noted that pre-dilution correction models are when the kinetic models are fit to describe the raw biomarker data without correcting for circuit dilution. Also, it should be noted that post-dilution correction models are when biomarker levels are corrected using perfusate exchange data to account for circuit dilution.

TABLE 10
Pre- and post-dilution correction mean R² comparison

| | Pre-dilution | Post-dilution | Paired comparison |
|---|---|---|---|
| Biomarker | correction R² | correction R² | (p-value) |
| GM-CSF | 0.7078 | 0.7998 | 0.00021 |
| IL-10 | 0.9119 | 0.9239 | <0.0001 |
| IL-1β | 0.7931 | 0.8736 | <0.0001 |
| IL-6 | 0.9421 | 0.9482 | <0.0001 |
| IL-8 | 0.9721 | 0.9747 | 0.044 |
| sTNFR1 | 0.7742 | 0.8929 | <0.0001 |
| sTREM1 | 0.8303 | 0.9278 | <0.0001 |

Paired samples Wilcoxon test

### Linear and Exponential Models are Robust to Random Reduction of Time Points for Clinical Translation:

The minimum number of time points required to establish a comparable model and the corresponding p-values are shown in Tables 11 and 12a-12d. Overall, linear and exponential models (In-transformed) showed better practicality for clinical translation as they used 2 out of 12 time points to establish a model that was representative of the model based on all time points (Table 12a and 12c). Interestingly, the exponential model without natural logarithm (In) transformation was generally more sensitive to the number of time points selected; in particular, the rate constant k was additionally challenging to model with less time points (Table 12d). Quadratic models generally required more time points to be selected (Table 12b). As for the 4PL and 5PL models, they generally used more than half of the original time points to establish comparable models. Due to the complexity of sigmoidal regression curves, 4PL and 5PL models were found to be the least practical models for clinical translation.

TABLE 11
Minimum number of time points required
to establish comparable models

| Model | Minimum number of time points required |
|---|---|
| Linear Model | randomly selecting 2 time points that are 15 mins apart |
| Quadratic Model | randomly selecting at least 3-5 time points that are 15 |
| | mins apart |
| Exponential | the required number of time points greatly vary |
| Model | |
| 4PL Model | requires more than 7 time points |
| 5PL Model | requires more than 7 time points |

Tables 12a-12d: Reducing the number of time points to establish comparable models. a) linear model; b) quadratic model; c) exponential model (In transformation applied); d) exponential model without In transformation.

12a)

Linear model - compare rate of change values

| Biomarker | Min # of time points | p-value |
|---|---|---|
| GM-CSF | 2 | 0.16 |
| IL-10 | 2 | 0.56 |
| IL-1β | 2 | 0.090 |
| IL-6 | 2 | 0.96 |
| IL-8 | 2 | 0.53 |
| sTNFR1 | 2 | 0.44 |
| sTREM1 | 2 | 0.76 |

Paired t-test

12b)
Paired t-test

| | Quadratic model - compare model coefficients | | | | | |
|---|---|---|---|---|---|---|
| Biomarker | a | p-value | b | p-value | c | p-value |
| GM-CSF | 3 | 0.10 | 3 | 0.64 | 3 | 0.6495 |
| IL-10 | 3 | 0.85 | 5 | 0.47 | >6 | <0.05 |
| IL-1β | 3 | 0.37 | 3 | 0.42 | 3 | 0.44 |
| IL-6 | 3 | 0.88 | 3 | 0.83 | 3 | 0.91 |
| IL-8 | 3 | 0.26 | 3 | 0.39 | 3 | 0.12 |
| sTNFR1 | 4 | 0.48 | 4 | 0.47 | 4 | 0.061 |
| sTREM1 | 3 | 0.76 | 3 | 0.52 | 3 | 0.34 |

12c)

Exponential model (In-transformed)-
compare rate of change values

| Biomarker | Min # of time points | p-value |
|---|---|---|
| GM-CSF | 2 | 0.76 |
| IL-10 | 2 | 0.44 |
| IL-1β | 2 | 0.39 |
| IL-6 | 2 | 0.68 |
| IL-8 | 2 | 0.71 |
| sTNFR1 | 2 | 0.37 |
| sTREM1 | 2 | 0.17 |

Paired t-test

12d)

| | Exponential model (no In transformation) - compare Y0 and k | | | |
|---|---|---|---|---|
| Biomarker | Y0 | p-value | k | p-value |
| GM-CSF | 2 | 0.069 | 6 | 0.063 |
| IL-10 | 2 | 0.37 | 2 | 0.050 |
| IL-1β | 2 | 0.22 | 2 | 0.12 |
| IL-6 | 4 | 0.13 | >6 | <0.01 |
| IL-8 | 2 | 0.33 | 2 | 0.29 |
| sTNFR1 | 2 | 0.085 | >6 | <0.01 |
| sTREM1 | 2 | 0.12 | >6 | <0.01 |

Paired t-test

Model features improved recipient outcome prediction versus biomarker data derived from single time point

Donor lung characteristics of 45 clinical EVLP cases are summarized in Table 13. Features derived from best-fit models for each of the seven biomarkers were first used as univariate features to predict ICU length of stay as binary classification (Tables 14a-14b). Compared to biomarker data measured at the point estimate (i.e., hourly), linear model features derived from GM-CSF, sTNFR1, and sTREM1 showed improvements in AUROC values (Table 14a). Specifically, rate of change values of the linear model improved prediction performance by 13% and 5% for GMCSF and sTNFR1, respectively. No improvement was associated with sTREM1 model features. Model features extracted from four interleukins (IL-10, IL-1β, IL-6, IL-8) also boosted AUROC values (Table 14b). For IL-1, rate of change and k increased AUROCs by 4% and 9%, respectively. Model features associated with IL-6 also led to a 9% increase in AUROC by Y0 and a 7% increase by k. Similarly, for IL-8, k boosted the AUROC value by 10%. No improvement was observed with IL-10 model features.

TABLE 13
Summary of donor lung characteristics (n = 45)

| Donor Characteristics | | |
|---|---|---|
| Donor Age (mean ± SD, years) | 47 ± 16 | |
| Donor Sex (%) | | |
| Male | 35 | (78%) |
| Female | 10 | (22%) |
| Cold Ischemic Time (mean ± SD, | 313 ± 79 | |
| years) | | |
| Donor Type (%) | | |
| DBD | 22 | (49%) |
| DCD | 23 | (51%) |
| EVLP Type (%) | | |
| Double lung | 41 | (91%) |
| Single lung | 4 | (9%) |
| EVLP outcome (%) | | |
| Double | 22 | (49%) |
| Single | 10 | (23%) |
| Declined | 13 | (29%) |

Tables 14a-14b: Univariate prediction AUROC results
using model features vs. single time point biomarker
value for ICU length of stay prediction. a) linear
model features; b) exponential model features
a)

| | Linear model features | | Hourly |
|---|---|---|---|
| | Rate of change | y-intercept | Data |
| GM-CSF | 73% | 52% | 60% |
| sTNFR1 | 58% | 52% | 53% |
| sTREM1 | 55% | 55% | 60% |

b)

| | Exponential model features | | | |
|---|---|---|---|---|
| | Rate of | | | Hourly |
| | change* | Y0 | k | Data |
| IL-10 | 59% | 55% | 47% | 59% |
| IL-1β | 54% | 45% | 58% | 49% |
| IL-6 | 49% | 64% | 62% | 55% |
| IL-8 | 55% | 54% | 63% | 53% |

*instantaneous rate of change

A multivariate logistic regression model combining all model features and 180-minute biomarker data for each of the seven biomarkers was employed to predict ICU length of stay (<3 days) (Table 15). Features extracted from best-fit models resulted in varying changes to biomarker prediction using AUROCs. Notably, kinetic model features derived from GM-CSF (AUROC=79% [57-99]), IL-10 (AUROC=81% [61-99]), and IL-8 (AUROC=72% [50-94]) dramatically improved the prediction of ICU length of stay.

TABLE 15
Multivariate prediction AUROC [95% CI] results combining
model features and single time point biomarker data for
all seven biomarkers for ICU length of stay prediction

| | All kinetic features | Hourly Data |
|---|---|---|
| GM-CSF | 79% [57-99] | 60% |
| sTNFR1 | 67% [42-92] | 53% |
| sTREM1 | 63% [37-88] | 60% |
| IL-10 | 81% [61-99] | 59% |
| IL-1ß | 61% [34-87] | 49% |
| IL-6 | 64% [39-89] | 55% |
| IL-8 | 72% [50-94] | 53% |

Note:
linear model features include rate of change and y-intercept values; exponential model features include instantaneous rate of change, Y0, and k values.

### Discussion

In study #2, the approach of using different mathematical models to describe unique temporal/kinetic behaviours of EVLP perfusate-derived protein biomarkers was successfully established. Adjusting measured biomarker levels for perfusate exchanges further revealed the true trend of change in biomarker kinetics and resulted in improved model fits and increased prognostic value of the biomarkers. More importantly, it was demonstrated that features extracted from best-fit perfusate models accurately reflect biomarker kinetics and improved prediction performance for lung transplant recipient outcomes. Univariate prediction using each of the model features led to an increase in the AUROC values compared to using just single time point biomarker level for prediction. A multivariate logistic regression model can combine all of the extracted feature values for the relevant model features and 180-min biomarker data also significantly improved prediction performance of ICU length of stay. The repeated sampling approach used in study #2 provided opportunities for more adaptive biomarker modeling as compared to the conventional hourly sampling.

Based on the model fit results, GM-CSF, sTNFR1, and sTREM1 can be well-described by a linear model, whereas IL-10, IL-1β, IL-6, IL-8 were better fit using an exponential growth curve. Similar trends of increase were also reported in a previous study looking at cytokine expression profile of human lungs during EVLP⁴⁹. EVLP-treated lungs exhibit endogenous capacity to produce inflammatory mediators. Previous study has shown that IL-6 and IL-8 derived from circulating perfusate exhibited more than 100-fold increase after 4 hours of EVLP; whereas sTNFR1 experienced a much lower increase over time⁷. This further validated the reliability of the results of study #2 and highlighted the importance of biomarker individuality. Accordingly, each biomarker may be modeled differently to fully reflect its unique behaviour.

Study #2 also demonstrated the predictive utility of biomarker kinetic model features, advancing the present understanding of conventional biomarker prediction which utilizes biomarker level measured at defined time points. Kinetic models are mainly associated with two advantages. Firstly, kinetic modeling has minimal dependence on EVLP duration as it mainly focuses on the trend of change over a given period of time. Secondly, kinetic modeling provides the opportunity for future research to include more model features with increasingly complex models. Overall, study #2 establishes a foundation to start exploring how to treat biomarkers differently in prediction models by tracking quantitative changes overtime.

The five kinetic models used in study #2 can be categorized into three groups: simple linear models, non-linear regression models (quadratic and exponential), and sigmoid curves (4PL and 5PL). As model complexity increases, increasing goodness of fit is expected as more explanatory terms are used to explain the variance within the data. Moreover, from the clinical translation perspective, models that not only well-describe the time series biomarker data were sought for, but such models should also be readily interpretable and practical for clinical translation.

The kinetic modeling presented in study #2 is more applicable for biomarkers that exhibit an obvious trend of accumulation. Additionally, the first hour of EVLP perfusion is considered as the “warming-up” phase where the perfusate flow rate and temperature are gradually increased to a required level¹. This protective perfusion strategy allows the donor lung to gradually reach physiologic state to minimize injury; however, this gradual process may result in partial release of certain biomarkers from individual lung regions, thereby hindering the kinetic model accuracy. Furthermore, the three-hour time window might not be enough to represent the complete kinetic profile of certain biomarkers since any delayed feedback response or unknown mechanistic accumulation pattern occurring during prolonged EVLP may potentially alter biomarker behaviour. While study #2 identified best-fit model for each biomarker, in at least one embodiment case-by-case variation in model fitting may be used since biomarkers related to each EVLP case may be treated differently based on case-specific characteristics.

Based on the results from study #2, it is believed that with current EVLP protocols, increasing density of data points by using shorter sampling time interval will improve modeling and prediction accuracy; and/or incorporation of advanced algorithm and automated repeated sampling throughout clinical EVLP may also provide opportunities for real-time analysis of biomarker trend and prediction model fitting. Furthermore, for EVLP perfusate biomarkers that are concentration-dependent biomarkers, it was demonstrated that applying a circuit dilution correction model improved the model fit for all seven biomarkers. It was also shown that features extracted from the best-fit kinetic models can be used to improve the prediction performance of recipient outcomes.

### Example 3—Study #3

A further study was performed on additional data “Test Dataset 2” from EVLP cases performed from December 2020 to August 2022, which was used to validate the machine learning approach. The study methods that were employed are the same as previously described for studies #1 and #2 with the difference being new/updated data from n=124 cases. The data from Test Dataset 2 were used in a similar way as Test Dataset 1. The trained InsighTx model was validated using data from Test Dataset 2. In other words, the InsighTx model was able to predict transplant outcomes using donor and EVLP features from Test Dataset 2. The kinetic modelling was not used in Test Dataset 2.

### Results

### Descriptive Data:

From 2008 to 2022, there were a total of n=725 eligible clinical EVLP cases that were included in InsighTx model development and validation. There were n=504 EVLP cases performed from 2008 to November 2019 that were used as a development dataset. Consecutive EVLP cases conducted between December 2019 to December 2020 (n=97) and December 2020 to August 2022 (n=124) were used as validation cohorts 1 and 2 respectively (Table 16). There were no significant differences in donor age, sex, BMI or type (Table 16); however, the proportion of donation after circulatory death (DCD) compared to donation after brain death (DBD) donors increased in the validation cohorts; median warm ischemic time was 65 minutes [IQR: 50-80 minutes]. Transplant rates and post-transplant outcomes significantly varied (Table 16). The rate of transplantation following EVLP was the highest in Test Dataset 1 (66%) and lowest in Test Dataset 2 (49%). While the incidence of PGD Grade 3 at 72 h was consistent in study #3, it was observed that the proportion of patients extubated in less than 72 h was highest in Test Dataset 1 (49%) and lowest in Test Dataset 2 (30%) (Table 16). Although extubation times varied, the median time spent in the ICU was similar across the datasets (Table 16). Of all donor lungs evaluated on EVLP, 38% resulted in transplantation and extubation in less than 72 h post-transplant, 22% were transplanted but associated with prolonged ventilation, and 40% were deemed unsuitable for transplant. These prevalence rates were used as the reference baseline for the area under the precision-recall curve (AUPRC) of EVLP and transplant outcomes.

### Performance Data:

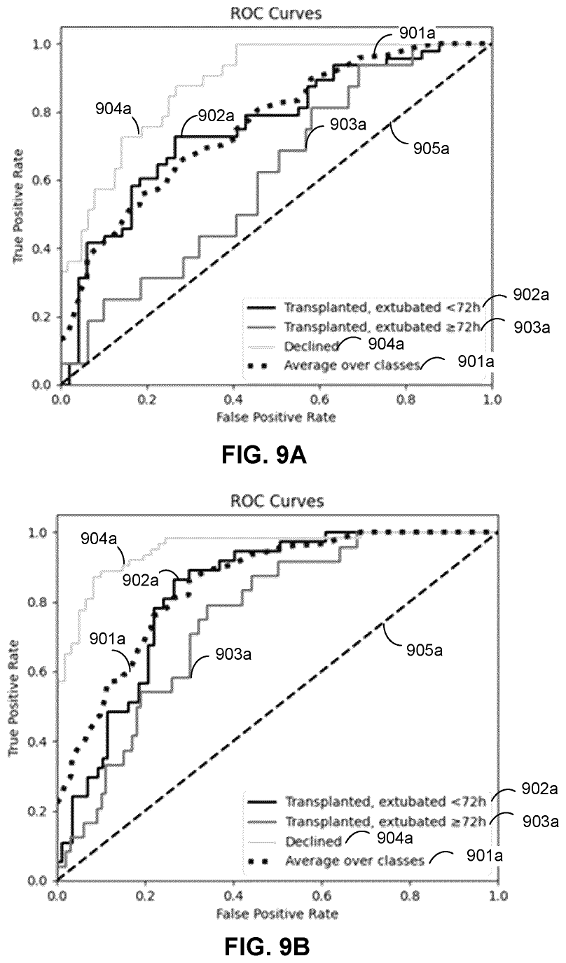

The overall insightTx model included features previously noted earlier in the description plus the new features discussed in this study. The AUROC for the overall InsighTx model was 79±3%, 75±4%, 85±3% in the training and test sets, respectively (Table 17 and FIGS. 9A-9B). Importantly, discrimination was high for identifying donor lungs on EVLP that resulted in a time to extubation less than 72 h (AUROC: 80±4% (training dataset), 76±6% (test dataset 1), 83±4% (test dataset 2)) and for identifying lungs that were unsuitable for transplantation (AUROC: 90±4% (training), 88±4% (test dataset 1), 95±2% (test dataset 2)). Although the prediction of prolonged time to extubation in transplant recipients was modest in test dataset 1 compared to the training dataset (AUROC: 67±6% (training dataset) vs. 62±9% (test dataset 1)), the overall InsighTx model performed well in test dataset 2 (AUROC: 76±6%) (Table 17). Importantly, the precision (positive predictive value) of the overall InsighTx model to identify any unsuitable donor lung (i.e., declined for transplant or extubated >72 h) was 81% and model precision for suitable donor lungs (i.e., extubated <72 h) was similar at 72%. Furthermore, the AUPRC showed a marked improvement of the overall InsighTx model to predict EVLP outcomes compared to baseline AUPRC values (prevalence of the respective endpoints). For patients extubated <72 h (baseline AUPRC 38%), the overall InsighTx model had an AUPRC of 67±6% in the training dataset, 74±8% in test dataset 1, and 64±10% in test dataset 2. Similar AUPRC results were observed in patients that required prolonged ventilation post-transplant: 40±7% (training dataset), 31±11% (test dataset 1), and 42±11% (test dataset 2) for the overall InsighTx model vs. 22% for the baseline AUPRC. Notably, the improvement in AUPRC was the strongest for lungs deemed unsuitable for transplant (overall InsighTx: 86±5% (training dataset), 81±7% (test dataset 1), and 96±2% (test dataset 2) vs. 40% baseline AUPRC). The relationship between the overall InsighTx model and PGD Grade 3 at 72h was further investigated. For donor lungs that were predicted to have a time to extubation <72 h using the overall InsighTx model, the negative predictive value (NPV) for PGD Grade 3 at 72h post-transplant was 88% [95% Cl: 84-91%, p<0.001, n=430].

TABLE 16
Clinical EVLP case characteristics for overall InsighTx model development

| | Training | | Test | | Test | | |
|---|---|---|---|---|---|---|---|
| | Dataset | | Dataset 1 | | Dataset 2 | | p-value |
| Date Range | 2008-2019 | | 2019-2020 | | 2020-2022 | | — |
| Number of Cases | 504 | | 97 | | 124 | | — |
| Mean Age (SD) - Years | 45 | (17) | 48 | (16) | 47 | (16) | 0.25 |
| Male Sex (%) | 328 | (65%) | 62 | (64%) | 79 | (64%) | 0.84 |
| Mean BMI (SD) | 27.3 | (6.5) | 27.3 | (6.0) | 28.7 | (7.1) | 0.69 |
| Donor Type DBD (%) | 259 | (51%) | 48 | (49%) | 49 | (40%) | 0.10 |
| EVLP Outcome | | | | | | | |
| Transplanted (%) | 313 | (62%) | 64 | (66%) | 61 | (49%) | 0.02 |
| Declined (%) | 191 | (38%) | 33 | (34%) | 63 | (51%) | |
| Transplant Outcome | | | | | | | |
| Extubated <72 h (%) | 190 | (38%) | 48 | (49%) | 37 | (30%) | 0.04 |
| PGD 3 at 72 h (%) | 59 | (12%) | 4 | (4%) | 12 | (10%) | 0.08 |
| Median ICU LOS [IQR] - Days | 4 | [2-10] | 4 | [2-6] | 5 | [3-11] | 0.17 |

Legend: SD = standard deviation; BMI = body mass index; DBD = donation after brain death; EVLP = ex vivo lung perfusion; PGD = primary graft dysfunction; ICU = intensive care unit; LOS = length of stay; IQR = interquartile range. Statistics: One-way ANOVA test for age and BMI; Kruskal-Wallis test for ICU LOS; Chi-square test for sex, donor type, EVLP outcome, PGD3, and extubation <72 h.

TABLE 17
AUROC performance of the overall InsighTx
model to predict EVLP and Tx outcomes

| | InsighTx | | | |
|---|---|---|---|---|
| | Model | Extuba- | Extuba- | Declined |
| AUROC (SD) | (Overall) | ted <72 h | ted ≥72 h | for Tx |
| Training Dataset | 79 (3) | 80 (4) | 67 (6) | 90 (4) |
| Test Dataset 1 | 75 (4) | 76 (6) | 62 (9) | 88 (4) |
| Test Dataset 2 | 85 (3) | 83 (4) | 76 (6) | 95 (2) |
| p-value¹ | p = 0.50 | p = 0.48 | p = 0.49 | p = 0.48 |
| p-value² | p = 0.36 | p = 0.33 | p = 0.46 | p = 0.32 |

Legend:
¹p-value for Test Dataset 1 vs. Training Dataset;
²p-value for Test Dataset 2 vs. Training Dataset;
AUROC = area under receiver operating characteristic curve (%); SD = standard deviation.

### Overall InsighTx Model Performance on Test Data:

Referring now to FIGS. 9A-9B, shown therein are the AUROC graphs for the overall InsighTx model performance in Test Dataset 1 (FIG. 9A) and Test Dataset 2 (FIG. 9B). The AUROCs for the overall InsighTx model (dotted blue line 901a, 901b), prediction of post-transplant extubation <72 h (black line 902a, 902b), ≥72 h (blue line 903a, 903b), and unsuitable for transplant (yellow line 904a, 904b). The dashed line 905a, 905b represents an AUROC of 50%.

### Adding Recipient Features to the ML Model Improves Performance:

In study #3 it was demonstrated that the donor-only model (InsighTx) can be further refined by adding recipient features. This allows for predictions to be personalized to a given recipient's physiological conditions. Accordingly, the InsighTx model can include at least one Recipient feature which may include, but are not limited to, one or more recipient physiological features and/or one or more recipient status features, for example. For instance, recipient features may include recipient age, recipient sex, recipient BMI, recipient status at assessment, listing and transplant admission and/or recipient indication.

### Methods

A random forest model was used to evaluate the addition of recipient physiological features (age, sex, body mass index (BMI), recipient status feature, and indication for transplant) to the outcome probabilities of the overall InsighTx model. The recipient status are categorical values that represent the severity of recipients' conditions and the urgency of transplant. These include: the recipient status at assessment, the recipient status at listing, and the recipient status at transplant admission. Values for recipient status features were recorded at assessment, listing, and transplant admission according to standard procedures at Toronto General Hospital (University Health Network, Toronto, ON). All EVLP cases that resulted in bilateral transplantation (n=368) were included, and five-fold cross validation was performed. Table 19 lists the summary statistics for recipient features used in this analysis.

### Results

### SUMMARY

The analysis included investigating whether the inclusion of certain recipient features increased the performance of the overall InsighTx model and the prediction of post-transplant time to extubation. To do this a sequential modeling approach was used where the results from the overall InsighTx model were combined with recipient age, recipient sex, recipient BMI, recipient status, and indication for transplant to generate a secondary, updated probability of post-transplant outcome. In other words, the sequential model takes the output probabilities of the InsighTx model (using features described herein from the donor only) and the recipient features as input variables to provide an updated probability prediction on post-transplant outcomes. As might be expected, the addition of at least one recipient feature increased the AUROC for the overall InsighTx model to discriminate which EVLP cases would result in short or prolonged time to extubation in transplant patients (Table 18). A significant increase of 10% in the AUROC was observed compared to a recipient-only model and a similar trend of +6% in AUROC was observed versus the InsighTx model alone (Table 18).

TABLE 18
Performance (AUROC) of donor and/or recipient models that
predict time to extubation in transplanted patients

| Model | AUROC (SD) |
|---|---|
| InsighTx + Recipient Features | 79 (4) |
| InsighTx | 73 (8) |
| Recipient Features Only | 69 (6) |
| p-value¹ | 0.17 |
| p-value² | 0.01 |

Legend:
¹p-value for “InsighTx + Recipient Features” vs. “InsighTx”;
²p-value for “InsighTx + Recipient Features” vs. “Recipient Features”;
AUROC = area under receiver operating characteristic curve (%); SD = standard deviation.

TABLE 19
Transplant patient characteristics for
InsighTx with recipient features model

| Number of Cases | 368 | |
|---|---|---|
| Mean Age (SD) - Years | 55 | (14) |
| Sex (%) | | |
| Female | 135 | (37%) |
| Male | 233 | (63%) |
| Mean BMI (SD) | 24 | (5) |
| Status at Assessment (%) | | |
| 1 | 197 | (53%) |
| 2 | 131 | (36%) |
| 3 | 40 | (11%) |
| Status at Listing (%) | | |
| 1 | 197 | (53%) |
| 2 | 130 | (36%) |
| 3 | 41 | (11%) |
| Status at Admission (%) | | |
| 1 | 116 | (32%) |
| 2 | 139 | (37%) |
| 3 | 113 | (31%) |
| Indication for Transplant (%) | | |
| PF/ILD/UIP/NSIP | 161 | (44%) |
| Emphysema/COPD | 131 | (36%) |
| Cystic Fibrosis | 43 | (12%) |
| Primary pulmonary | 15 | (4%) |
| hypertension | | |
| Retransplant | 16 | (4%) |
| Other | 2 | (<1%) |

Legend: SD = standard deviation; BMI = body mass index; PF = pulmonary fibrosis; ILD = interstitial lung disease; UIP = usual interstitial pneumonia; NSIP = nonspecific interstitial pneumonia; COPD = chronic obstructive pulmonary disease.

### Real-Time Ventilator Data Extraction and Analysis

### Summary

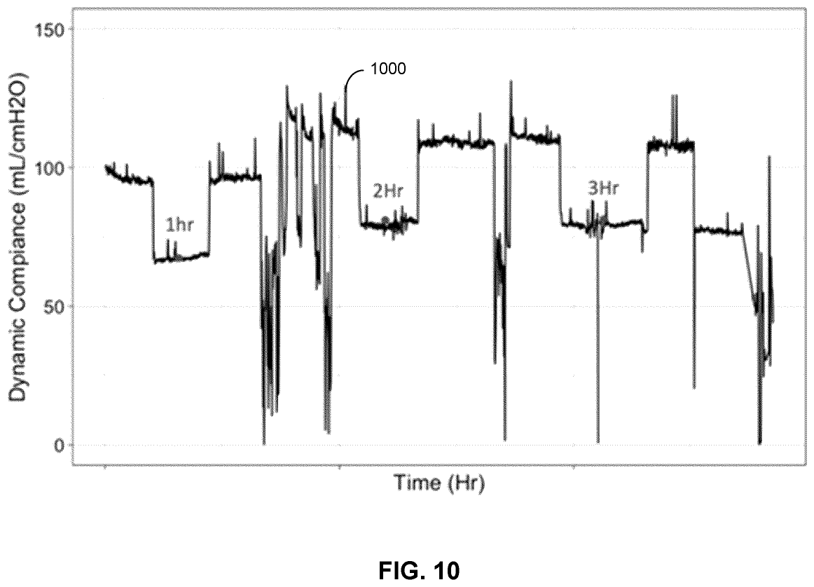

High-resolution flow and pressure data was recorded from an ICU-grade ventilator during a human EVLP case (use of the term human here is meant to exclude animal EVLP). Using this highly resolved time series data, software code was developed, as described below, to construct the ventilation waveform and perform continuous, breath-by-breath analysis of lung function parameters, such as dynamic compliance (FIG. 10). This data was used to form a more complete picture of lung ventilation during EVLP, which provides additional biomarkers and can be used to better understand patterns & trends in the longitudinal data (e.g., protein kinetics).

### Methods

Pressure and flow data were recorded at 100 Hz from an ICU-grade ventilator (Maquet Servo-i, Siemens Healthineers). Software programs for self-defined functions were written using the R Programming Language for raw file conversion, breath segmentation, and breath feature extraction. The start and end timestamps associated with each auto-recognized breath also served as unique identifiers of each breath. Moreover, interventional events performed during EVLP ventilation such as inspiratory pauses and hourly EVLP assessments were also recognized using self-defined functions (this is code written to achieve a specific task of the analysis) that were included in the software program to further assist the high-level analysis of EVLP ventilation and its association with outcomes.

For example, a script may be written in a programming language, such as the R programming language, to read and analyze the pressure and flow data recorded from the ventilator. The analysis of the pressure and flow data may include: raw file conversion (where files are converted to a readable format, for example, a CSV file), breath segmentation (where the timeseries data is divided into individual breaths based on the physiological patterns of breath cycles), and breath feature extraction (where respiratory parameters such as dynamic compliance, for example, are extracted from breath cycles using mathematical calculations). Inspiratory pauses and EVLP assessments are both recognized based on the unique changes in behaviours of the breath cycle due to clinical intervention during EVLP.

Each flow-controlled breath cycle starts with delivering a constant flow of gas to inflate the lungs. This process is also associated with an increase in pressure as the lung inflates. At the end of inspiration flow and pressure both drop as the lungs recoil due to its intrinsic tendency to deflate following inflation. The physiological patterns for inspiration and expiration refer to how the flow and pressure traces change as the lungs inflate and deflate in every breath cycle and are used to perform breath segmentation after breath parameter extraction involves determining values for one or more of the following breath parameters: Inspiratory time, expiratory time, PEEP (positive end-expiratory pressure), Peak pressure, Mean pressure, Plateau pressure, Inspiratory volume, Expiratory volume, Dynamic compliance, Static compliance and/or Stress index. These features can either be directly read on the breath waveform or can be calculated based on standard physiological definitions. The physiological definitions of these features are well-established.

### Results

### Example Data:

Referring now to FIG. 10, shown therein is real-time ventilator data captured during human EVLP. Breath-by-breath recording and analysis of dynamic compliance measurements (black line 1000) are shown compared to the data derived from the traditional approach of hourly recording (red dots at 1HR, 2HR and 3HR. As can be seen there is quite a variation in compliance measurements that are not captured by the traditional hourly recording time points.

### Automated Recognition of Important Segments of Ventilator Data:

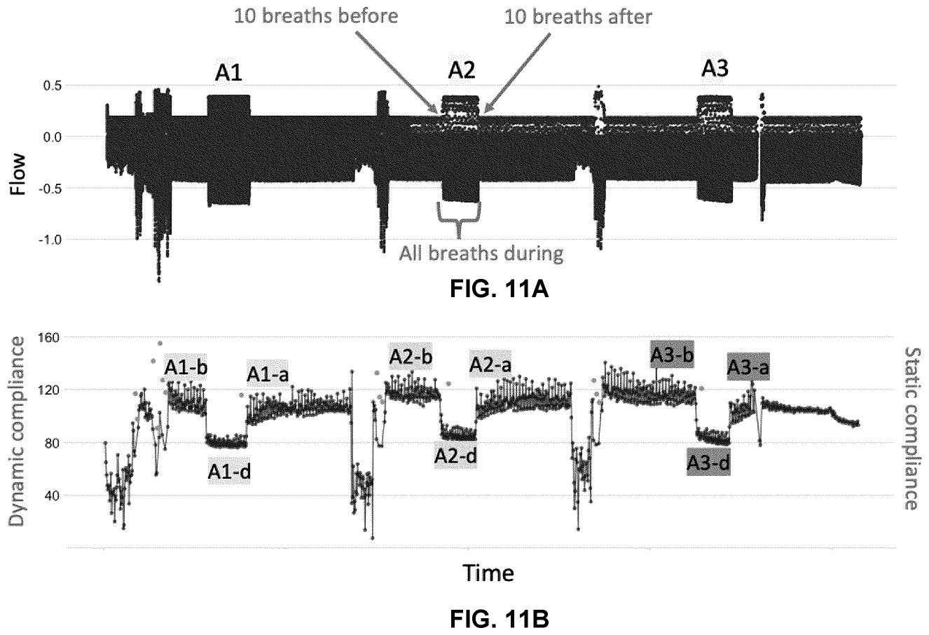

Referring now to FIG. 11A, Ventilator flow versus time is shown with annotations for three lung assessments performed during EVLP (A1, A2, A3). Referring now to FIG. 11B, dynamic compliance versus time is shown with annotations for individual breath segments recorded before (“b”), during (“d”), and after (“a”) lung assessments performed during EVLP. The dots indicate static compliance values from inspiratory pauses during EVLP.

Real-Time (High Resolution) Ventilator Data Features are Associated with Patient Outcomes:

Data from n=50 EVLP cases where ‘good’ outcomes are defined as time to extubation <72 h and ‘poor’ outcomes are defined as time to extubation more than 72 h or lungs deemed unsuitable for transplantation were assessed to determine whether ventilator data features may be associated with patient outcomes and hence serve as new predictive biomarkers that may be used in the InsighTx model.

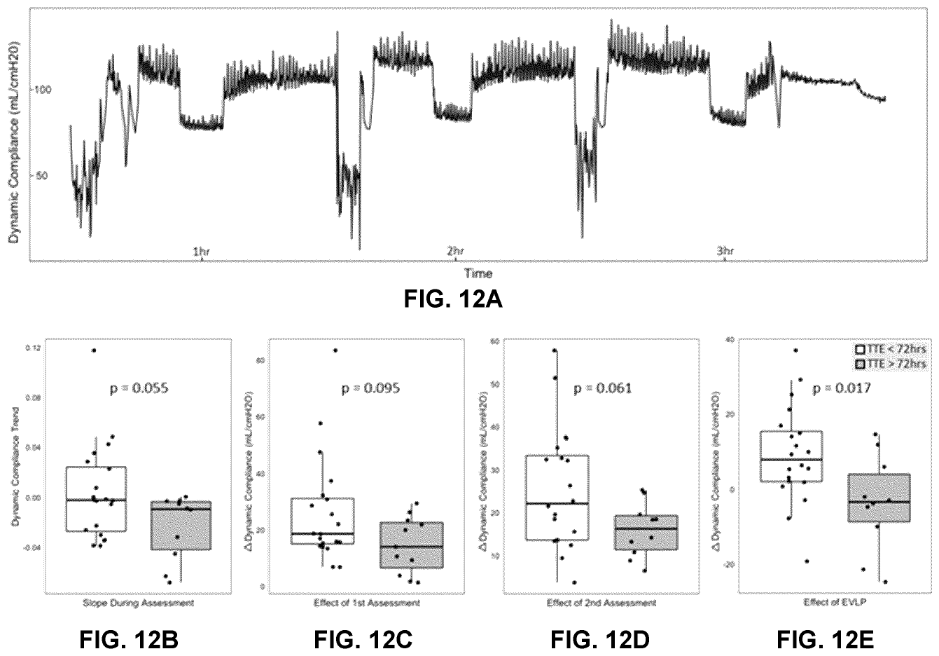

Referring now to FIGS. 12A-12E, shown therein is an example of the results of breath-by-breath ventilator analysis. FIG. 12A provides an example plot of breath-by-breath dynamic compliance over time. FIG. 12B shows a comparison of breath-by-breath dynamic compliance trend value during assessment vs. recipient outcome (TTE<72 hrs). FIG. 12C shows a comparison of changes in breath-by-breath dynamic compliance in a first EVLP assessment vs. recipient outcome (TTE<72 hrs). FIG. 12D shows a comparison of changes in breath-by-breath dynamic compliance in a second EVLP assessment vs. recipient outcome (TTE<72 hrs). FIG. 12E shows a comparison of changes in breath-by-breath dynamic compliance from the start to the end of EVLP vs. recipient outcome (TTE<72 hrs).

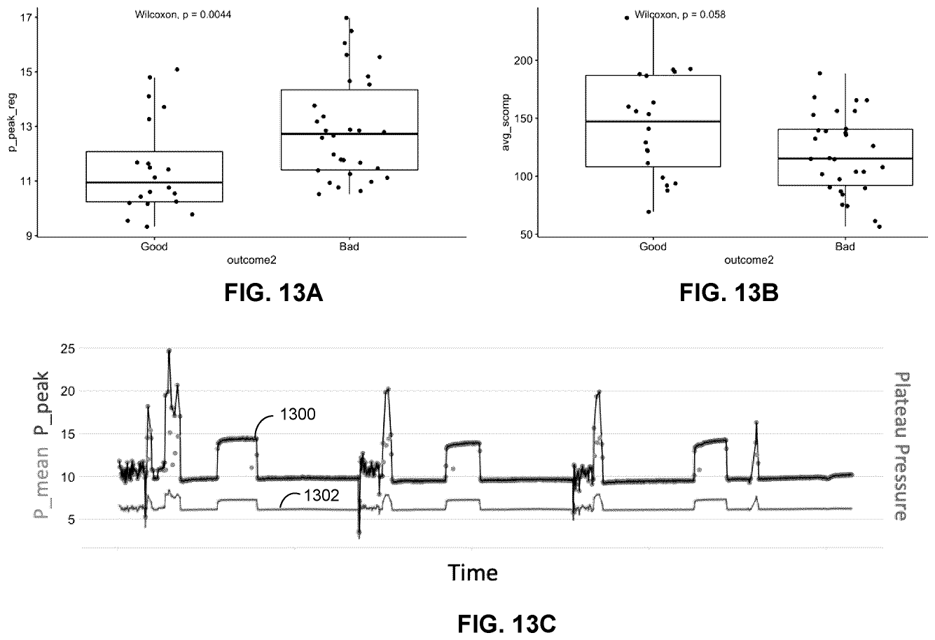

Referring now to FIGS. 13A-13C, shown therein is an example using airway pressures. In particular, mean peak pressure from donor lung breaths during EVLP (FIG. 13A) and mean static compliance, (FIG. 13B) are shown in good (TTE<72 hrs) and poor (TTE>72 hrs+declined) outcome groups. FIG. 13C shows breath-by-breath mean pressure 1300 and peak pressure 1302 on the y-axis vs. time on the x-axis. The dots show the plateau pressure from every inspiratory pause performed during EVLP.

### Real-Time Blood Gas Measurements:

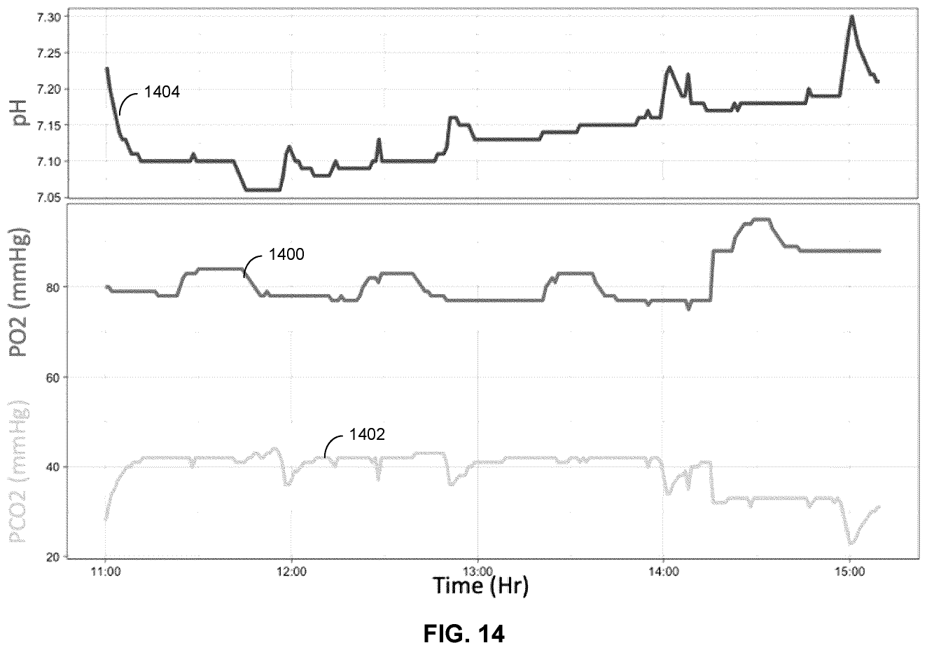

High-resolution data representative of lung physiology (i.e., pO2, pCO2) and biochemistry (i.e., pH values, electrolytes) were acquired using a blood parameter monitor designed for cardiopulmonary bypass surgery (CDI Blood Parameter Monitoring System 550, Terumo Cardiovascular), An example of this data is shown in FIG. 14.

### Methods

The CDI550 real-time blood parameter monitoring system was connected in parallel with the ex vivo lung perfusion system on both the left atrial (LA) and pulmonary artery (PA) side. The single-use in-line sensors anchored on LA and PA sides provide real-time monitoring of pH, PCO2, and PO2, and potassium every six seconds. The LA sensor can be connected between the LA line near the dome and the recirculation line, whereas the PA sensor can be more conveniently placed in the sampling line. The sensors are designed to use specifically with the CDI550 monitor. They are clinical-grade and commercially available as is known by those skilled in the art.

### Results

Referring now to FIG. 14, shown therein is an example of pilot real-time data recording in lung perfusate using a porcine model of EVLP. Real-time data extraction of EVLP perfusate features using the CD1550 monitor to quantify: partial pressure of oxygen (pO₂) (trace 1400) and carbon dioxide (pCO₂) (trace 1402), and perfusate pH (blue trace 1404).

### Discussion

FIG. 10-13 demonstrate the use of real-time ventilator flow and pressure data to aid in detailed analysis of individual breaths during clinical EVLP. Physiological features extracted by breath-by-breath analysis provide further understanding of lung physiology during EVLP and present association with post-transplant outcomes. FIG. 14 presents an example of real-time monitoring and recording of important clinical parameters during EVLP.

Table 16 describes the clinical EVLP cases in the study cohort, including basic donor information as well as EVLP and post-transplant outcomes. Table 17 contains model performances in AUROC from different datasets classifications. FIGS. 9A and 9B specifically show ROC curves from the AUROC performances of Test Datasets 1 and 2. Furthermore, the model performances with recipient information are in Table 18, while the transplant patient characteristics for the model with recipient features are described in Table 19.

While the applicant's teachings described herein are in conjunction with various embodiments for illustrative purposes, it is not intended that the applicant's teachings be limited to such embodiments. On the contrary, the embodiments of the present disclosure described above are intended to be examples only and it is not intended that the applicant's teachings be limited to such embodiments. The applicant's teachings described and illustrated herein encompass various alternatives, modifications, and equivalents, without generally departing from the embodiments described herein. While the systems, devices, and processes disclosed and shown herein may comprise a specific number of elements/components, the systems, devices, and assemblies may be modified to include additional or fewer of such elements/components as is known to those skilled in the art. For example, selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology. The entire disclosures of all references recited above are incorporated herein by reference.

### REFERENCES

1. Cypel M, Yeung J C, Liu M, Anraku M, Chen F, Karolak W, Sato M, Laratta J, Azad S, Madonik M, Chow C W, Chaparro C, Hutcheon M, Singer L G, Slutsky A S, Yasufuku K, de Perrot M, Pierre A F, Waddell T K, Keshavjee S. Normothermic ex vivo lung perfusion in clinical lung transplantation. N Engl J Med. 2011 Apr. 14; 364(15):1431-40. doi: 10.1056/NEJMoa1014597. PMID: 21488765.
2. Ceresa C D L, Nasralla D, Pollok J M, Friend P J. Machine perfusion of the liver: applications in transplantation and beyond. Nat Rev Gastroenterol Hepatol. 2022 Jan. 7. doi: 10.1038/s41575-021-00557-8. Epub ahead of print.
3. Ardehali A, Esmailian F, Deng M, Soltesz E, Hsich E, Naka Y, Mancini D, Camacho M, Zucker M, Leprince P, Padera R, Kobashigawa J; PROCEED II trial investigators. Ex-vivo perfusion of donor hearts for human heart transplantation (PROCEED II): a prospective, open-label, multicentre, randomised non-inferiority trial. Lancet. 2015 Jun. 27; 385(9987):2577-84. doi: 10.1016/S0140-6736(15)60261-6. Epub 2015 Apr. 14. PMID: 25888086.
4. Kaths J M, Spetzler V N, Goldaracena N, Echeverri J, Louis K S, Foltys D B, Strempel M, Yip P, John R, Mucsi I, Ghanekar A, Bagli D, Robinson L, Selzner M. Normothermic Ex Vivo Kidney Perfusion for the Preservation of Kidney Grafts prior to Transplantation. J Vis Exp. 2015 Jul. 15; (101):e52909. doi: 10.3791/52909. PMID: 26275014; PMCID: PMC4544894.
5. Prudhomme T, Mulvey J F, Young L A J, Mesnard B, Lo Faro M L, Ogbemudia A E, Dengu F, Friend P J, Ploeg R, Hunter J P, Branchereau J. Ischemia-Reperfusion Injuries Assessment during Pancreas Preservation. Int J Mol Sci. 2021 May 13; 22(10):5172. doi: 10.3390/ijms22105172. PMID: 34068301; PMCID: PMC8153272.
6. Whitson B A, Black S M. Organ assessment and repair centers: The future of transplantation is near. World J Transplant. 2014 Jun. 24; 4(2):40-2. doi: 10.5500/wjt.v4.i2.40. PMID: 25032094; PMCID: PMC4094951.
7. Keshavjee, S. Human organ repair centers: Fact or fiction?. JTCVS. 2020 Sep. 1; 3:164-168.
8. Cypel M, Yeung J C, Hirayama S, Rubacha M, Fischer S, Anraku M, Sato M, Harwood S, Pierre A, Waddell T K, de Perrot M, Liu M, Keshavjee S. Technique for prolonged normothermic ex vivo lung perfusion. J Heart Lung Transplant. 2008 Dec.; 27(12):1319-25. doi: 10.1016/j.healun.2008.09.003. PMID: 19059112.
9. Divithotawela C, Cypel M, Martinu T, Singer L G, Binnie M, Chow C W, Chaparro C, Waddell T K, de Perrot M, Pierre A, Yasufuku K, Yeung J C, Donahoe L, Keshavjee S, Tikkanen J M. Long-term Outcomes of Lung Transplant With Ex Vivo Lung Perfusion. JAMA Surg. 2019 Dec. 1; 154(12):1143-1150. doi: 10.1001/jamasurg.2019.4079. PMID: 31596484; PMCID: PMC6802423.
10. Suarez-Pierre A, Choudhury R, Carroll A M, King R W, Iguidbashian J, Cotton J, Colborn K L, Kennealey P T, Cleveland J C, Pomfret E, Fullerton D A. Measuring the effect of the COVID-19 pandemic on solid organ transplantation. Am J Surg. 2021 Dec. 30:S0002-9610(21)00784-4. doi: 10.1016/j.amjsurg.2021.12.036. Epub ahead of print. PMID: 34980465; PMCID: PMC8717917.
11. Watanabe T, Cypel M, Keshavjee S. Ex vivo lung perfusion. J Thorac Dis. 2021 Nov.; 13(11):6602-6617. doi: 10.21037/jtd-2021-23. PMID: 34992839; PMCID: PMC8662477.
12. Okahara S, Levvey B, McDonald M, D'Costa R, Opdam H, Pilcher D V, Snell G I. Common Criteria for Ex Vivo Lung Perfusion Have No Significant Impact on Posttransplant Outcomes. Ann Thorac Surg. 2021 April; 111(4):1156-1163. doi: 10.1016/j.athoracsur.2020.06.081. Epub 2020 Sep. 2. PMID: 32890490.
13. Possoz J, Neyrinck A, Van Raemdonck D. Ex vivo lung perfusion prior to transplantation: an overview of current clinical practice worldwide. J Thorac Dis. 2019 April; 11(4):1635-1650. doi: 10.21037/jtd.2019.04.33. PMID: 31179109; PMCID: PMC6531747.
14. Di Nardo M, Del Sorbo L, Sage A, Ma J, Liu M, Yeung J C, Valero J, Ghany R, Cypel M, Keshavjee S. Predicting donor lung acceptance for transplant during ex vivo lung perfusion: The E X vivo lung Perfusion pREdiction (EXPIRE). Am J Transplant. 2021 Nov.; 21(11):3704-3713. doi: 10.1111/ajt.16616. Epub 2021 Jun. 11. PMID: 33872459.
15. Sage A T, Richard-Greenblatt M, Zhong K, Bai X H, Snow M B, Babits M, Ali A, Baciu C, Yeung J C, Liu M, Cypel M, Kain K C, Keshavjee S. Prediction of donor related lung injury in clinical lung transplantation using a validated ex vivo lung perfusion inflammation score. J Heart Lung Transplant. 2021 July; 40(7):687-695. doi: 10.1016/j.healun.2021.03.002. Epub 2021 Mar. 5. PMID: 33781664.
16. Ferdinand J R, Morrison M I, Andreasson A, Charlton C, Chhatwal A K, Scott W E 3rd, Borthwick L A, Clatworthy M R, Fisher A J. Transcriptional analysis identifies potential novel biomarkers associated with successful ex-vivo perfusion of human donor lungs. Clin Transplant. 2021 Dec. 26:e14570. doi: 10.1111/ctr.14570. Epub ahead of print. PMID: 34954872.
17. Andreasson A S I, Borthwick L A, Gillespie C, Jiwa K, Scott J, Henderson P, Mayes J, Romano R, Roman M, Ali S, Fildes J E, Marczin N, Dark J H, Fisher A J; DEVELOP-U K Investigators. The role of interleukin-1β as a predictive biomarker and potential therapeutic target during clinical ex vivo lung perfusion. J Heart Lung Transplant. 2017 September; 36(9):985-995. doi: 10.1016/j.healun.2017.05.012. Epub 2017 May 12. PMID: 28551353; PMCID: PMC5578478.
18. Andreasson A S, Karamanou D M, Gillespie C S, Özalp F, Butt T, Hill P, Jiwa K, Walden H R, Green N J, Borthwick L A, Clark S C, Pauli H, Gould K F, Corris P A, Ali S, Dark J H, Fisher A J. Profiling inflammation and tissue injury markers in perfusate and bronchoalveolar lavage fluid during human ex vivo lung perfusion. Eur J Cardiothorac Surg. 2017 Mar. 1; 51(3):577-586. doi: 10.1093/ejcts/ezw358. PMID: 28082471; PMCID: PMC5400024.
19. Chen T, Guestrin C. XGBoost: A Scalable Tree Boosting System. Proceedings of the 22nd ACM SIGKDD International Conference on Knowledge Discovery and Data Mining (KDD '16). 2016 785-794.
20. Machuca T N, Cypel M, Yeung J C, Bonato R, Zamel R, Chen M, Azad S, Hsin M K, Saito T, Guan Z, Waddell T K, Liu M, Keshavjee S. Protein expression profiling predicts graft performance in clinical ex vivo lung perfusion. Ann Surg. 2015 March; 261(3):591-7. doi: 10.1097/SLA.0000000000000974. PMID: 25371129.
21. Machuca T N, Cypel M, Zhao Y, Grasemann H, Tavasoli F, Yeung J C, Bonato R, Chen M, Zamel R, Chun Y M, Guan Z, de Perrot M, Waddell T K, Liu M, Keshavjee S. The role of the endothelin-1 pathway as a biomarker for donor lung assessment in clinical ex vivo lung perfusion. 50 J Heart Lung Transplant. 2015 June; 34(6):849-57. doi: 10.1016/j.healun.2015.01.003. Epub 2015 Jan 16. PMID: 25907141.
22. Hashimoto K, Cypel M, Kim H, Machuca T N, Nakajima D, Chen M, Hsin M K, Zamel R, Azad S, Waddell T K, Liu M, Keshavjee S. Soluble Adhesion Molecules During Ex Vivo Lung Perfusion Are Associated With Posttransplant Primary Graft Dysfunction. Am J Transplant. 2017 May; 17(5):1396-1404. doi: 10.1111/ajt.14160. Epub 2017 Feb. 1. PMID: 27977885.
23. Hashimoto K, Cypel M, Juvet S, Saito T, Zamel R, Machuca T N, Hsin M, Kim H, Waddell T K, Liu M, Keshavjee S. Higher M30 and high mobility group box 1 protein levels in ex vivo lung perfusate are associated with primary graft dysfunction after human lung transplantation. J Heart Lung Transplant. 2017 Jun. 21:S1053-2498(17)31870-3. doi: 10.1016/j.healun.2017.06.005. Epub ahead of print. PMID: 28689646.
24. Caldarone L, Mariscal A, Sage A, Khan M, Juvet S, Martinu T, Zamel R, Cypel M, Liu M, Palaniyar N, Keshavjee S. Neutrophil extracellular traps in ex vivo lung perfusion perfusate predict the clinical outcome of lung transplant recipients. Eur Respir J. 2019 Apr. 4; 53(4):1801736. doi: 10.1183/13993003.01736-2018. PMID: 30655281.
25. Kanou T, Nakahira K, Choi A M, Yeung J C, Cypel M, Liu M, Keshavjee S. Cell-free DNA in human ex vivo lung perfusate as a potential biomarker to predict the risk of primary graft dysfunction in lung transplantation. J Thorac Cardiovasc Surg. 2021 August; 162(2):490-499.e2. doi: 10.1016/j.jtcvs.2020.08.008. Epub 2020 Aug. 11. PMID: 32928548.
26. Lee J C, Diamond J M, Christie J D. Critical care management of the lung transplant recipient. Curr Respir Care Rep. 2012; 1(3):168-176. doi: 10.1007/s13665-012-0018-9. Epub 2012 Jun. 22. PMID: 32288970; PMCID: PMC7102351.
27. Clausen E, Cantu E. Primary graft dysfunction: what we know. J Thorac Dis. 2021 Nov; 13(11):6618-6627. doi: 10.21037/jtd-2021-18. PMID: 34992840; PMCID: PMC8662499.
28. Abrams D, Agerstrand C, Beitler J R, Karagiannidis C, Madahar P, Yip N H, Pesenti A, Slutsky A S, Brochard L, Brodie D. Risks and Benefits of Ultra-Lung-Protective Invasive Mechanical Ventilation Strategies with a Focus on Extracorporeal Support. Am J Respir Crit Care Med. 2022 Jan. 19. doi: 10.1164/rccm.202110-2252C P. Epub ahead of print. PMID: 35044901.
29. Bellini V, Valente M, Del Rio P, Bignami E. Artificial intelligence in thoracic surgery: a narrative review. J Thorac Dis. 2021 December; 13(12):6963-6975. doi: 10.21037/jtd-21-761. PMID: 35070380; PMCID:PMC8743413.
30. Cypel M, Yeung J C, Hirayama S, et al. Technique for Prolonged Normothermic Ex Vivo Lung Perfusion. The Journal of Heart and Lung Transplantation. 2008; 27(12):1319-1325. doi:10.1016/j.healun.2008.09.003.
31. Cypel M, Yeung J C, Machuca T, et al. Experience with the first 50 ex vivo lung perfusions in clinical transplantation. The Journal of Thoracic and Cardiovascular Surgery. 2012; 144(5):1200-1207. doi:10.1016/j.jtcvs.2012.08.009.
32. Boffini M, Ricci D, Barbero C, et al. Ex Vivo Lung Perfusion Increases the Pool of Lung Grafts: Analysis of Its Potential and Real Impact on a Lung Transplant Program. Transplantation Proceedings. 2013; 45(7):2624-2626. doi:10.1016/j.transproceed.2013.08.004.
33. Yeung J C, Cypel M, Machuca T N, et al. Physiologic assessment of the ex vivo donor lung for transplantation. The Journal of Heart and Lung Transplantation. 2012; 31(10):1120-1126. doi:10.1016/j.healun.2012.08.016.
34. Yeung J C, Cypel M, Keshavjee S. Ex-vivo lung perfusion: the model for the organ reconditioning hub. Current Opinion in Organ Transplantation. 2017; 22(3):287-289. doi:10.1097/MOT.0000000000000404.
35. Hsin M K, Zamel R, Cypel M, et al. Metabolic Profile of Ex Vivo Lung Perfusate Yields Biomarkers for Lung Transplant Outcomes. Annals of Surgery. 2018; 267(1):196-197. doi:10.1097/SLA.0000000000002016.
36. Leligdowicz A, Ross J T, Nesseler N, Matthay M A. The endogenous capacity to produce proinflammatory mediators by the ex vivo human perfused lung. ICMx. 2020; 8(1):56. doi:10.1186/s40635-020-00343-x.
37. Kanou T, Nakahira K, Choi A M, et al. Cell-free DNA in human ex vivo lung perfusate as a potential biomarker to predict the risk of primary graft dysfunction in lung transplantation. The Journal of Thoracic and Cardiovascular Surgery. 2021; 162(2):490-499.e2. doi:10.1016/j.jtcvs.2020.08.008.
38. Shin J, Hsin M K, Baciu C, et al. Use of metabolomics to identify strategies to improve and prolong ex vivo lung perfusion for lung transplants. The Journal of Heart and Lung Transplantation. 2021; 40(6):525-535. doi:10.1016/j.healun.2021.02.002.
39. Machuca T N, Cypel M, Yeung J C, et al. Protein Expression Profiling Predicts Graft Performance in Clinical Ex Vivo Lung Perfusion. Annals of Surgery. 2015; 261(3):591-597. doi:10.1097/SLA.0000000000000974.
40. Andreasson A S I, Karamanou D M, Gillespie C S, et al. Profiling inflammation and tissue injury markers in perfusate and bronchoalveolar lavage fluid during human ex vivo lung perfusion. Eur J Cardiothorac Surg. Published online Jan. 12, 2017:ezw358. doi:10.1093/ejcts/ezw358.
41. Sage A T, Richard-Greenblatt M, Zhong K, et al. Prediction of donor related lung injury in clinical lung transplantation using a validated ex vivo lung perfusion inflammation score. The Journal of Heart and Lung Transplantation. Published online March 2021:S1053249821022191. doi:10.1016/j.healun.2021.03.002.
42. Rösler B, Herold S. Lung epithelial G M-CSF improves host defense function and epithelial repair in influenza virus pneumonia-α new therapeutic strategy?Mol Cell Pediatr. 2016; 3(1):29. doi:10.1186/s40348-016-0055-5.
43. Michlewska S, Dransfield I, Megson I L, Rossi A G. Macrophage phagocytosis of apoptotic neutrophils is critically regulated by the opposing actions of pro-inflammatory and anti-inflammatory agents: key role for TNF-α. FASEB j. 2009; 23(3):844-854. doi:10.1096/fj.08-121228.
44. Lecube A, Sampol G, Muñoz X, Ferrer R, Hernández C, Simó R. TNF-α system and lung function impairment in obesity. Cytokine. 2011; 54(2):121-124. doi:10.1016/j.cyto.2011.01.010.
45. Baciu C, Sage A, Zamel R, et al. Transcriptomic investigation reveals donor specific gene signatures in human lung transplants. Eur Respir J. Published online Jan. 1, 2020:2000327. doi:10.1183/13993003.00327-2020.
46. Shi J X, Li J S, Hu R, et al. Diagnostic Value of sTREM-1 in Bronchoalveolar Lavage Fluid in ICU Patients With Bacterial Lung Infections: A Bivariate Meta-Analysis. Rojas M, ed. PLoS ONE. 2013; 8(5):e65436. doi:10.1371/journal.pone.0065436.
47. Wright S W, Lovelace-Macon L, Hantrakun V, et al. sTREM-1 predicts mortality in hospitalized patients with infection in a tropical, middle-income country. BMC Med. 2020; 18(1):159. doi:10.1186/s12916-020-01627-5.
48. Mason S J, Graham N E. Areas beneath the relative operating characteristics (ROC) and relative operating levels (ROL) curves: Statistical significance and interpretation. Q J R Meteorol Soc. 2002; 128(584):2145-2166. doi:10.1256/003590002320603584.
49. Sadaria M R, Smith P D, Fullerton D A, et al. Cytokine Expression Profile in Human Lungs Undergoing Normothermic Ex-Vivo Lung Perfusion. The Annals of Thoracic Surgery. 2011; 92(2):478-484. doi:10.1016/j.athoracsur.2011.04.027.

1. A method for predicting an outcome for a lung undergoing an ex vivo lung perfusion (EVLP), the method comprising:
obtaining EVLP data for measuring at least one lung feature of the lung taken over a time period;
measuring values from the EVLP data to obtain at least one time series for the at least one lung feature over the time period;
fitting the at least one time series of the at least one lung feature with a corresponding lung feature model, and determining values for lung feature model parameters that define the at least one corresponding lung feature model based on said fitting; and
calculating a prediction of an outcome for an individual who receives the lung with a machine learning model, wherein the values for the lung feature model parameters are used as inputs to the machine learning model and the machine learning model outputs the prediction of the outcome.

2. The method of claim 1, wherein the at least one lung feature comprises at least one biomarker and the corresponding lung feature model is a corresponding biomarker model.

3. The method of claim 2, further comprising filtering the measured values of the at least one biomarker to account for circuit dilution prior to the step of fitting the time series of the measured values of the at least one biomarker with the corresponding biomarker models.

4. (canceled)

5. The method of claim 2, wherein the at least one biomarker comprises GM-CSF, IL-10, IL-1β, IL-6, IL-8, sTNFR1, and/or sTREM1.

6. The method of claim 2, further comprising using standardized perfusate data to correct the measured values of the at least one biomarkers.

7. The method of claim 2, wherein; (a) the corresponding biomarker model comprises a linear model, a quadratic model, an exponential model, a 4PL model, or a 5PL model; and/or (b) the machine learning model comprises a univariate or multivariate logistic regression model.

8. (canceled)

9. The method of claim 1, wherein the outcome comprises: (a) an EVLP outcome including suitable or unsuitable, (b) a transplant outcome including good patient outcome or bad patient outcome and/or (c) ICU length of stay.

10. (canceled)

11. (canceled)

12. (canceled)

13. A method for predicting an outcome for a lung undergoing an ex vivo lung perfusion (EVLP), the method comprising:
obtaining values for a first set of features from data obtained for lung features including one or more donor parameters, one or more physiological parameters, one or more biochemical parameters, and/or one or more biomarkers, where at least a portion of the data was collected during EVLP;
processing the data for a subset of the parameters to determine values for a second set of features based on temporal characteristics of the data for the subset of the parameters; and
determining predicted probabilities for at least one outcome classification by providing the values for the first and second sets of features as inputs to a machine learning prediction model.

14. The method of claim 13, wherein the one or more donor parameters comprise: age; sex; body mass index (BMI); donor type donation-after-brain-death (DBD); donor total lung capacity (TLC) and/or donation-after-cardiac-death (DCD).

15. The method of claim 13, wherein the first set of features also include one or more recipient parameters.

16. The method of claim 15, wherein the one or more recipient parameters comprise: recipient age, recipient sex, recipient BMI, recipient status, and/or indication for transplant.

17. The method of claim 13, wherein the one or more physiological parameters comprise: change in oxygen partial pressure (ΔPO2); change in carbon dioxide partial pressure (ΔPCO2); pH; ventilator air flow, dynamic compliance; static compliance; pulmonary artery (PA) & left atrial (LA) pressure; vascular resistance; airway pressure including peak, mean and plateau; positive end-expiratory pressure (PEEP); edema; perfusate loss; and/or +/−exchange.

18. The method of claim 13, wherein the one or more biochemical parameters comprise: Ca²⁺; Cl⁻; K⁺; Na⁺; base excess; HCO₃⁻; pH; glucose; and/or lactate.

19. The method of claim 13, wherein the one or more biomarkers comprise: GM-CSF; IL-10; IL-1β; IL-6; IL-8, sTNFR1, and/or sTREM1.

20. The method of claim 13, wherein the temporal characteristics comprise statistical measurements including a minimum value, a maximum value, a last recorded value and/or a trend for the data collected for the subset of the parameters.

21. The method of claim 13, wherein values for at least one of the features from the first set of features are determined by; measuring values from the data to obtain at least one time series for the at least one lung feature over a time period; fitting the at least one time series for the at least one lung feature with a corresponding lung feature model, determining values for lung feature model parameters that define the at least one corresponding lung feature model based on said fitting, and providing the values for the lung feature model parameters as input to the machine learning prediction model.

22. The method of claim 13, wherein values for at least one of the features from the first set of features are determined by obtaining an x-ray image of the lung, performing image processing on the x-ray image and determining the values from the processed x-ray image.

23. The method of claim 13, wherein the machine learning model outputs at least one of three-outcome classifications comprising: (i) lung unsuitable for transplantation; (ii) EVLP transplant resulting in a time to extubation of >72 hours; and (iii) EVLP transplant resulting in a time to extubation of <72 hours.

24. The method of claim 13, wherein the machine learning model comprises a decision tree algorithm or a extreme gradient boosting (XGBoost) machine learning algorithm.

25. (canceled)

26. The method claim 13, wherein the machine learning model determines a relative weighting of the values for the first and second sets of features.

27. (canceled)

28. The method of claim 13, wherein the machine learning model is trained using k-fold cross-validation.

29. (canceled)

30. (canceled)

31. An electronic device for predicting an outcome for a lung undergoing an ex vivo lung perfusion (EVLP), wherein the electronic device comprises:
one or more user interfaces for receiving user input and providing indication to the user;
a memory for storing program instructions; and
a processor being communicatively coupled to the memory and the one or more user interfaces, the processor, when executing the program instructions, being configured to perform the method according to claim 13.

32. (canceled)
